(19) United States
(12) Patent Application Publication
(10) Pub. No.: US 2024/0050600 A1
(43) Pub. Date: Feb. 15, 2024

(54) Radiopharmaceuticals, uses thereof, and methods for the production thereof

(71) Applicant: Clarity Pharmaceuticals Ltd, Eveleigh, NSW (AU)

(72) Inventors: Lachlan Eion MCINNES, Eveleigh, NSW (AU); Paul Stephen DONNELLY, Eveleigh, NSW (AU); Ellen Marianne VAN DAM, Eveleigh, NSW (AU); Matthew John HARRIS, Eveleigh, NSW (AU)

(73) Assignee: Clarity Pharmaceuticals Ltd, Eveleigh, NSW (AU)

(21) Appl. No.: 18/021,252

(22) PCT Filed: Aug. 13, 2021
(86) PCT No.: PCT/AU2021/050896

§ 371 Date: Feb. 14, 2023

(30) Foreign Application Priority Data
Aug. 14, 2020 (AU) 2020902889

(51) Int. Cl. A61K 51/08; A61P 35/00

(52) U.S. Cl. CPC A61K 51/088; A61P 35/00

(57) ABSTRACT

Radiopharmaceutical compounds of general Formula (I) are based on a sarcophagine cage having a PEG linker-group X through to a group Y capable of binding to a biological receptor and an albumin-binding group Z for the treatment, diagnosis or imaging of diseases including cancer, and the production of such compounds and their complexes.

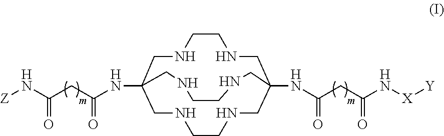

### FIELD

The present invention relates to compounds that have more than one group having physiological functionality and can also complex a radionuclide. The compounds may be useful as radiopharmaceuticals for the treatment, diagnosis and imaging of diseases such as cancer. The present invention also relates to methods for the production of such compounds and their complexes.

### BACKGROUND

Compounds that may be radiolabeled with a radionuclide are often used as radiopharmaceuticals for the diagnosis and/or treatment of diseases such as cancer. In addition to being suitable for radiolabeling, the compound itself must show sufficient binding at specific sites that are characteristic for the indication that is diagnosed or treated. Where the compound does not bind in concentrations effective for diagnosis or treatment, increasing the dose of the compound administered may be an option, however this is associated with increased expense, exposure to radioactivity and other adverse physiological effects.

Once administered to a subject, the compound moves through the circulation and either binds to the target site or is eliminated. Where the compound shows limited or slower binding to the target site, the majority of the compound may be eliminated and cleared from the subject such that the amount that is in fact bound to the target site is insufficient for diagnosis or treatment. While the dose or amount of the compound administered may be increased in an attempt to provide increased binding, this also increases the likelihood of unwanted off-target effects (e.g. radiation damage at other sites).

Radiopharmaceuticals often contain multiple functional groups, for example, at least a group capable of coordinating the radionuclide and a separate group capable of binding to the target site. Given the presence of such groups on the same compound, there are various synthetic challenges to overcome when producing such a compound. This includes issues related to inherent stability, solubility and reactivity of the fragments or reagents required for synthesis of the compounds. Even if a particular compound containing the desired fragments may be synthesized, the overall yields of the compounds are often low. Given the complexity of such compounds, difficulties with purification may also be encountered.

Since the compounds are intended to deliver a radioisotope to the target site for diagnosis or therapy, the radioisotope should be securely coordinated by the compound in order to reduce the loss of the radioisotope. Where this occurs, radiation damage to unwanted sites (where the radioisotope persists in the circulation of the subject) may occur. Since radioisotopes undergo spontaneous decay, the products of decay may also lead to undesirable damage of the compound coordinating the radioisotope.

Accordingly, there is a need for compounds suitable for use as radiopharmaceuticals where the compounds allow for sufficient binding to the target site, while still having the requisite stability for administration as a radiopharmaceutical. There is also the need for processes that allow for such compounds to be produced in sufficient amounts.

### SUMMARY OF THE INVENTION

According to a first aspect, the present invention provides a compound of Formula (I), a salt, complex, isomer, solvate or prodrug thereof:

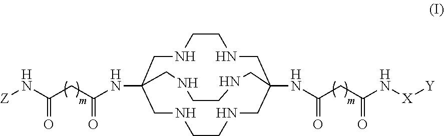

wherein:
each m is independently an integer from 1 to 10;
X is a linker having the formula

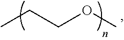

where n is an integer from 1 to 10;
Y is a group capable of binding to a biological receptor; and
Z is an albumin-binding group.

In an embodiment, m is the same in each case. In other embodiments, m is different in each case. In an embodiment, the m is independently an integer from 1 to 6. In another embodiment, m is independently an integer from 2 to 5. In a preferred embodiment, m in each case is the same and is 3.

According to a second aspect, the present invention provides a compound of Formula (I) having the structure of Formula (Ia), a salt, complex, isomer, solvate or prodrug thereof:

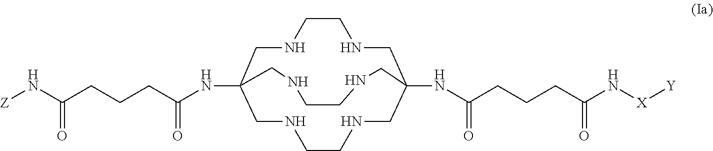

wherein:
X is a linker having the formula

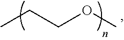

where n is an integer from 1 to 10;
Y is a group capable of binding to a biological receptor; and
Z is an albumin-binding group.

In an embodiment of the above aspects, Z is an albumin-binding group of Formula (II):

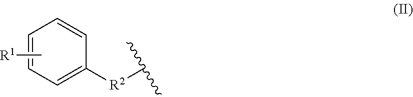

wherein:
R¹ is an optionally substituted alkyl group, optionally substituted alkoxy group, halogen or CN group; and
R² is an optionally substituted C₂-C₁₆ alkylene group, wherein one or more alkylene units in the alkylene group may be replaced with a group selected from a urea, thiourea, amine, amide, carbonyl, heteroatom, polyethylene oxide or arylene group, wherein the arylene group may be optionally substituted.

In an embodiment, Z is an albumin-binding group of Formula (II′):

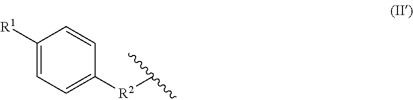

wherein R¹ and R² are as defined above.

In an embodiment, Z is an albumin-binding group of Formula (IIa′):

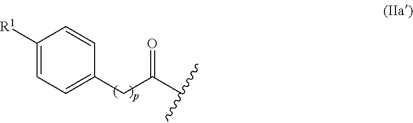

wherein p is an integer selected from 1 to 6 and R¹ is as defined above.

In another embodiment, Z is an albumin-binding group of Formula (IIb′):

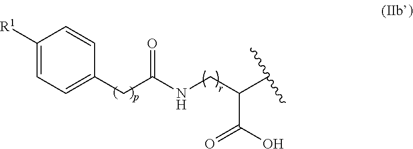

wherein p and r are an integer independently selected from 1 to 6 and R¹ is as defined above.

In an embodiment of Formula (II) and subformulae thereof, R¹ is an optionally substituted C₁-C₁₂ alkyl group, optionally substituted C₁-C₂₀ alkoxy group, halogen or CN group.

In another embodiment of Formula (II), R² is an optionally substituted C₂-C₁₆ alkylene group, wherein one alkylene unit is replaced with a carbonyl group. In another embodiment, R² is an optionally substituted C₂-C₁₆ group, wherein one alkylene unit is replaced with an amide group. In another embodiment, R² is an optionally substituted C₂-C₁₆ alkylene group, wherein one alkylene unit is replaced with a carbonyl group and one alkylene unit is replaced with an amide group. In another embodiment, R² is an optionally substituted C₂-C₁₆ alkylene group, wherein one or more alkylene unit is replaced with one or more polyethylene oxide groups.

In an embodiment, the polyethylene oxide group has the following structure and may replace an alkylene unit in R² such that the atoms of the polyethylene oxide group are joined from either direction:

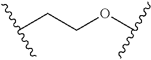 [SMILES: *CCO*]

In an embodiment of the above aspects, Z is an albumin-binding group that is an optionally substituted C₂-C₂₀ alkyl group, where one or more alkylene units may be replaced with a group selected from a urea, thiourea, amine, amide, carbonyl, heteroatom or a polyethylene oxide group.

In an embodiment, Z is a C₁₀-C₂₂ alkyl group. In another embodiment, Z is a C₁₀-C₂₂ alkyl group where one or more alkylene units is replaced with a carbonyl group. In another embodiment, Z is a C₁₀-C₂₂ alkyl group where one or more alkylene units is replaced with a carboxylic acid group. In another embodiment, Z is a C₁₀-C₂₂ alkyl group where one or more alkylene units is replaced with a polyethylene oxide group. In another embodiment, Z is a C₁₀-C₂₂ alkyl group where one or more alkylene groups is replaced with an amide group. In another embodiment, Z is a C₁₀-C₂₂ alkyl group having a terminal carboxylic acid group.

Further embodiments of Z are depicted below:

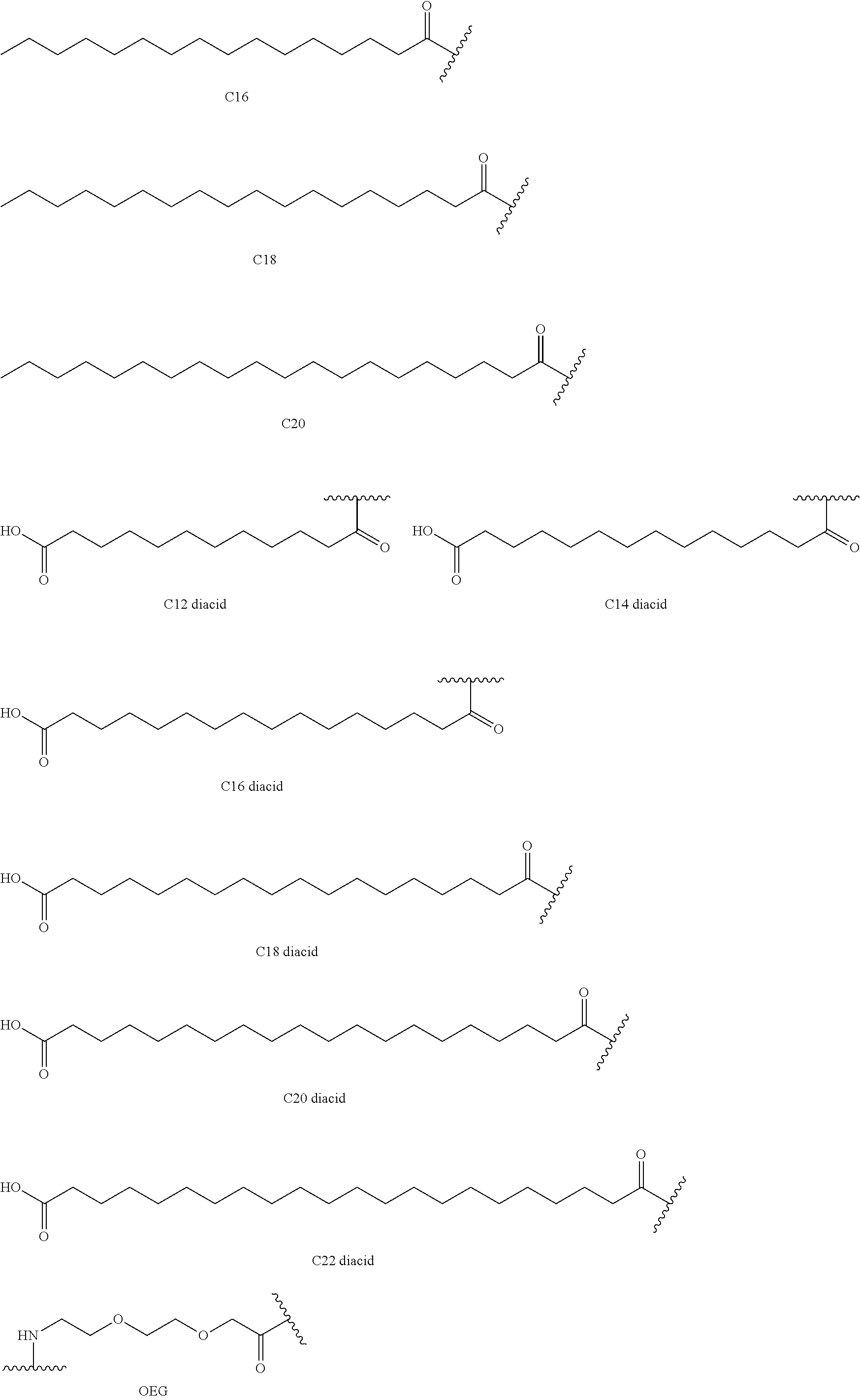

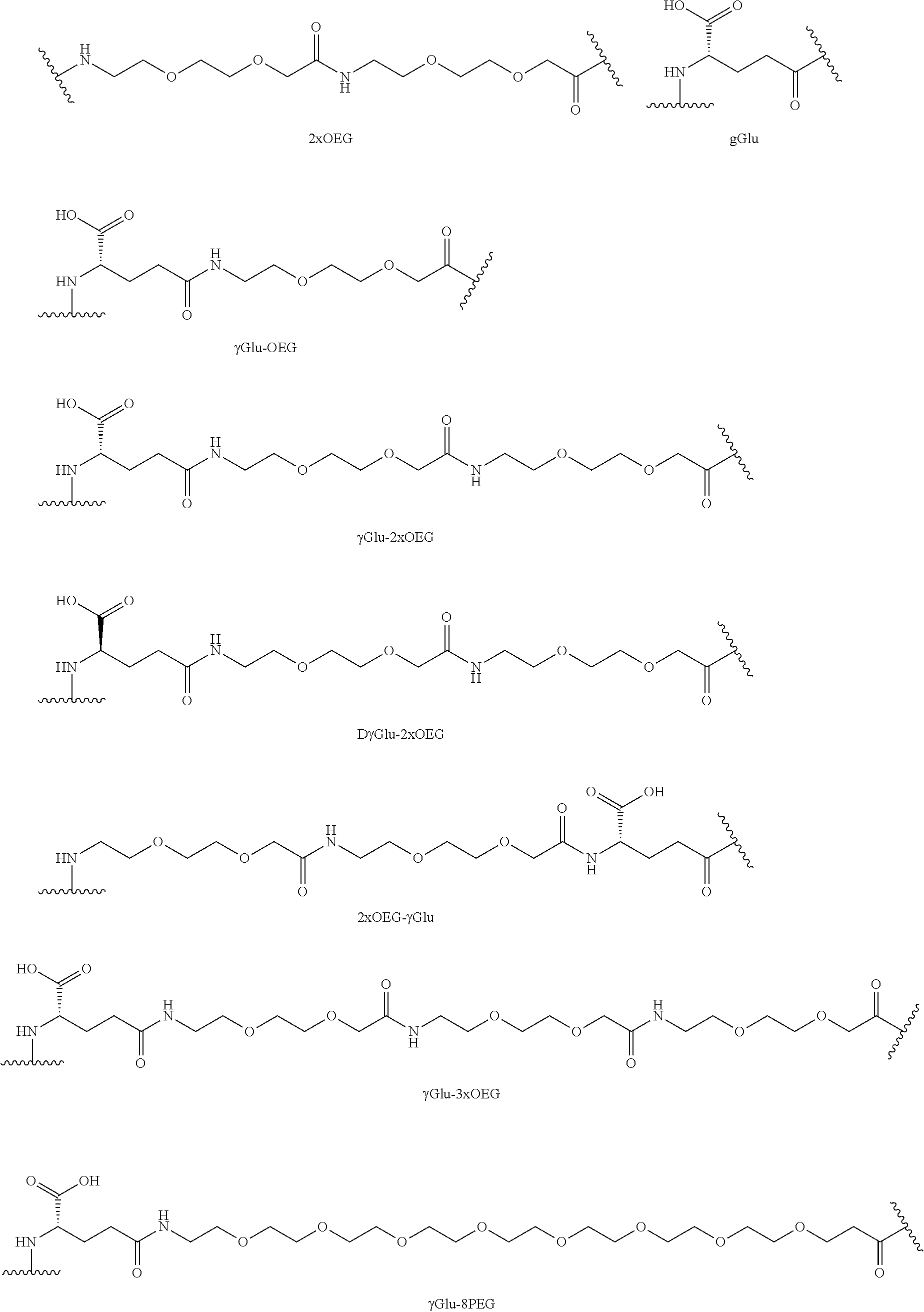

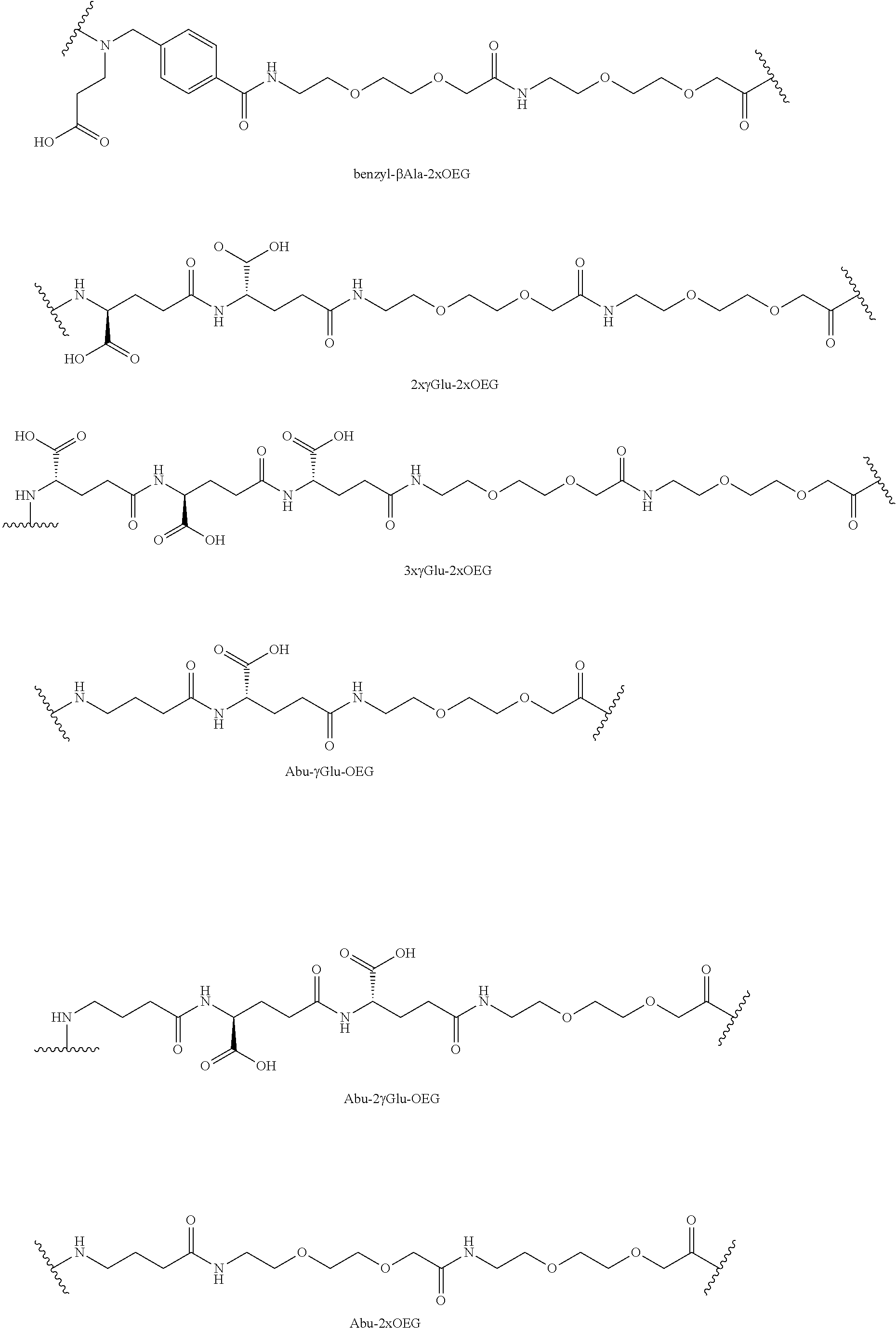

In an embodiment, R¹ is a C₁-C₁₂ alkyl group. In another embodiment, R¹ is a C₁-C₂₀ alkoxy group. In a preferred embodiment, R¹ is a C₁ alkyl or C₁ alkoxy group. In another preferred embodiment, R¹ is halogen group, particularly I or Cl. In another preferred embodiment, R¹ is a CN group.

In other embodiments, R² is an optionally substituted C₂-C₁₀ alkylene group. In another embodiment, R² is an optionally substituted C₂-C₄ alkylene group. In other embodiments, one alkylene group of R² is replaced with a urea, thiourea, amine, amide, carbonyl or heteroatom group. In another embodiment, R² is a C₂-C₁₀ alkylene group optionally substituted by a carboxylic acid group.

In some embodiments, the group Z of Formula (II) has one of the following structures:

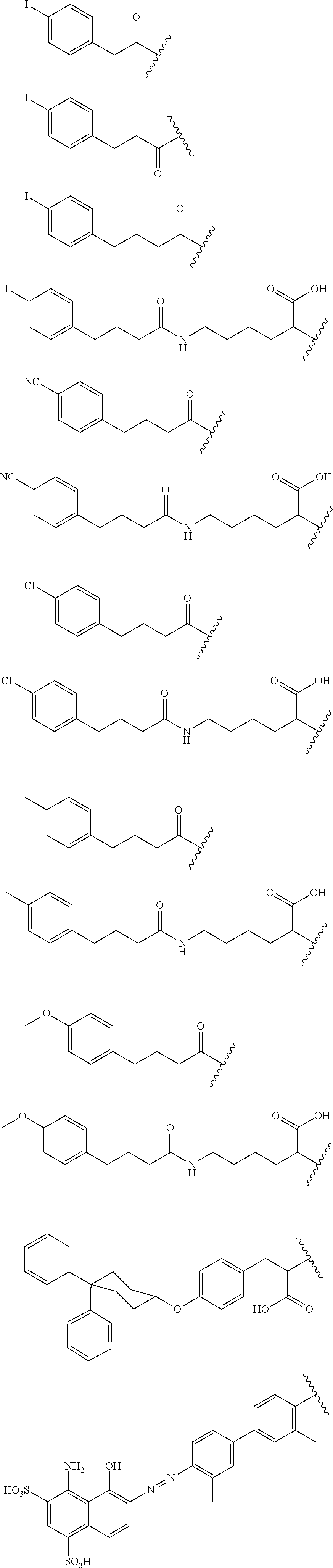

In an embodiment, the compound according to the first or second aspects is coordinated with a metal ion. In a further embodiment, the metal ion is a radionuclide of Cu. In a preferred embodiment, the radionuclide of Cu is selected from the group consisting of ⁶⁰Cu, ⁶¹Cu, ⁶²Cu, ⁶⁴Cu and ⁶⁷Cu.

According to a third aspect, the present invention provides a composition comprising a compound as defined in the first or second aspects, and one or more pharmaceutically acceptable excipients.

According to a fourth aspect, the present invention provides a process for producing a compound of Formula (I) or a salt, complex, isomer, solvate or protected form thereof:

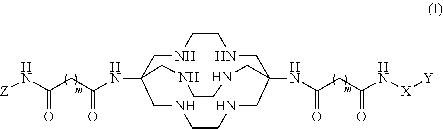

wherein m, X, Y and Z are as defined in the first aspect, the process comprising the steps of:
i) coupling a compound of Formula (IV), or a salt, complex, isomer or solvate thereof,

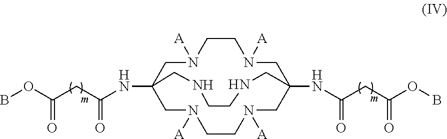

with a compound of Formula (V) or a salt thereof,

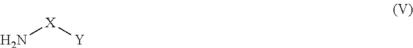

for a time and under conditions to give a compound of Formula (VI) or a salt thereof

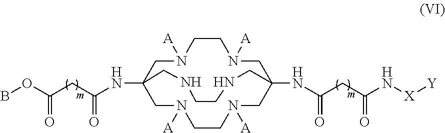

wherein A is a nitrogen-protecting group and B is an oxygen-protecting group;
ii) coupling a compound of Formula (VI) of step i) with a compound of Formula (VII) or a salt thereof:

H₂N—Z (VII)

for a time and under conditions to give a compound of Formula (VIII) or a salt thereof

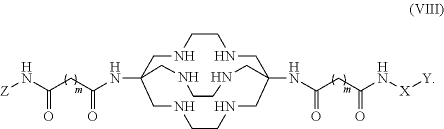

In an embodiment, m is the same in each case. In other embodiments, m is different in each case. In an embodiment, the m is independently an integer from 1 to 6. In another embodiment, m is independently an integer from 2 to 5. In a preferred embodiment, m in each case is the same and is 3.

In an embodiment, the process of the fourth aspect provides a compound of Formula (Ia) or a salt, complex, isomer, solvate or protected form thereof:

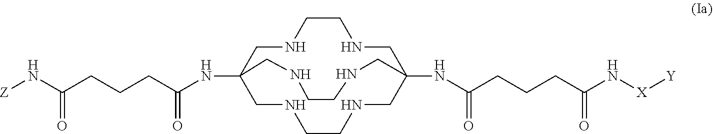

wherein X, Y and Z are as defined in the second aspect, the process comprising the steps of:
i) coupling a compound of Formula (IVa), or a salt, complex, isomer or solvate thereof,

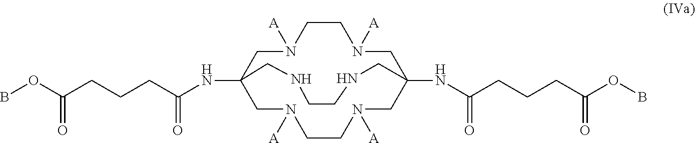

with a compound of Formula (Va) or a salt thereof,

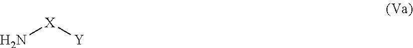

for a time and under conditions to give a compound of Formula (VIa) or a salt thereof

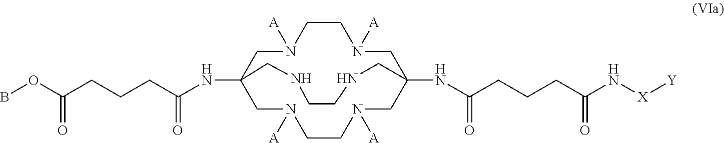

wherein A is a nitrogen-protecting group and B is an oxygen-protecting group;
ii) coupling a compound of Formula (VIa) of step i) with a compound of Formula (VIIa) or a salt thereof:

H₂N—Z (VIIa)
for a time and under conditions to give a compound of Formula (VIIIa) or a salt thereof

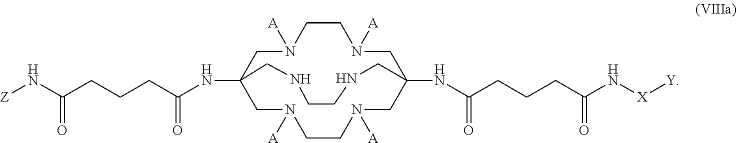

In an embodiment, the process according to the fourth aspect is performed under microwave conditions.

According to a fifth aspect, the present invention provides a method for treating a cancer in a subject, the method comprising administering to a subject in need thereof a compound of Formula (I) or Formula (Ia) as defined in the first or second aspects, respectively, wherein the compound is complexed with a radioisotope.

According to a sixth aspect, the present invention provides a method of radioimaging a subject, the method comprising administering to a subject in need thereof a compound of Formula (I) or Formula (Ia) as defined the first or second aspects, respectively, wherein the compound is complexed with a radioisotope.

According to a seventh aspect, the present invention provides the use of a compound of Formula (I) or Formula (Ia) as defined in the first aspect or second aspects, respectively, in the manufacture of a medicament for treating a cancer.

### BRIEF DESCRIPTION OF THE FIGURES

FIG. 1: RadioHPLC trace of [⁶⁴Cu]CuSar(ABG)(BBN), showing the radiochemical purity of the radiolabelled compound.

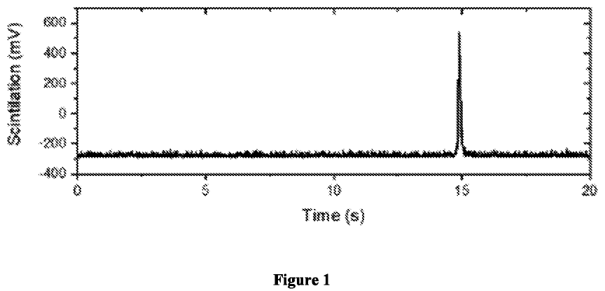

FIG. 2. Representative radioTLC trace of purified peptide showing successful labelling with ⁶⁴Cu. Two radioTLCs were run for each sample, without 50 mM EDTA (left) and with 50 mM EDTA (right). Without EDTA, both the radiolabelled peptide and unbound radioisotope are represented at ˜50 mm. With EDTA, the radiolabelled peptide is represented at ˜120 mm, while the unbound radioisotope is represented at ˜160 mm. Both radioTLCs show that there is negligible unbound ⁶⁴Cu.

FIG. 3. Axial, coronal and sagittal PET-CT projections of a representative mouse injected with Sar(ABG)(BBN) radiolabelled with ⁶⁴Cu at 1 hour.

FIG. 4. Axial, coronal and sagittal PET-CT projections of a representative mouse injected with Sar(ABG)(BBN) radiolabelled with ⁶⁴Cu at 4 hours.

FIG. 5. Axial, coronal and sagittal PET-CT projections of a representative mouse injected with Sar(ABG)(BBN) radiolabelled with ⁶⁴Cu at 24 hours.

FIG. 6. 3D reconstruction of PET-CT projections of a representative mouse injected with Sar(ABG)(BBN) at radiolabelled with ⁶⁴Cu 24 hours.

FIG. 7. Biodistribution of injected peptide after 24 hours, as determined by identifying regions of interest from PET images obtained of mice injected with the relevant peptides radiolabelled with 64Cu.

### DETAILED DESCRIPTION

The present invention provides compounds of Formula (I), salts, complexes, isomers, solvates, prodrugs and protected forms thereof:

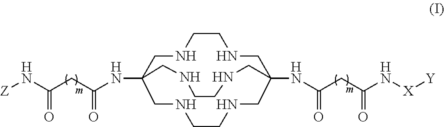

wherein:
m is independently an integer from 1 to 10;
X is a linker having the formula

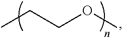

where n is an integer from 1 to 10;
Y is a group capable of binding to a biological receptor; and
Z is an albumin-binding group.

In some embodiments, the present invention also provides compounds of Formula (I) having the structure of Formula (Ia):

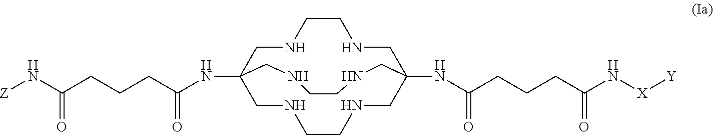

wherein:
X is a linker having the formula

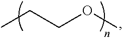

where n is an integer from 1 to 10;
Y is a group capable of binding to a biological receptor; and
Z is an albumin-binding group.

As used herein, the term “salt” refers to acid addition salts and base addition salts of the compound, where the salt is prepared from an inorganic or organic acid, or an inorganic or organic base. In some embodiments, the salts of the compounds of the present invention may be pharmaceutically acceptable salts.

As used herein, the term “pharmaceutically acceptable salts” refers to salts that retain the desired biological activity of the above-identified compounds and may also be acid addition salts or base addition salts. Suitable pharmaceutically acceptable acid addition salts of compounds of Formula (I) may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic, arylsulfonic. Additional information on pharmaceutically acceptable salts can be found in Remington's Pharmaceutical Sciences, 19th Edition, Mack Publishing Co., Easton, PA 1995. In the case of agents that are solids, it is understood by those skilled in the art that the inventive compounds, agents and salts may exist in different crystalline or polymorphic forms, all of which are intended to be within the scope of the present invention and specified formulae.

As used herein, the term “complex” refers to a compound that is then coordinated by a metal ion.

As used herein, the term “solvate” refers to a complex of the compound, where the complex may have variable stoichiometry formed by a solute and a solvent. Such solvents in the solvate should not interfere with the biological activity of the solute. Examples of suitable solvents may include water, ethanol or acetic acid. Methods of solvation of the compound are generally known in the art.

As used herein, the term “prodrug” refers to and includes derivatives that are converted in vivo to the compounds of the present invention. Such derivatives would readily occur to those skilled in the art, and include, for example, compounds containing a free hydroxyl group that is converted into an ester derivative, or containing a ring nitrogen atom that is converted to an N-oxide. Examples of ester derivatives include alkyl esters, phosphate esters and those formed from amino acids.

As used herein, the term “isomer” refers to and includes all stereoisomers of the compounds of the present invention. Examples of isomers include diastereomers and enantiomers, where appropriate.

The compounds of Formula (I) contain a nitrogen-containing macrocycle, which is capable of chelating metal ions. The macrocycle of Formula (I) is a 3,6,10,13,16,19-hexaazabicyclo[6.6.6]icosane and may be referred to as a “sarcophagine”. The sarcophagines of Formula (I) contain six nitrogen atoms, where one or more of the nitrogen atoms may be protected with a suitable protecting group.

The present inventors have found that compounds of Formula (I) containing a group capable of binding to serum albumin, i.e. Z in a compound of Formula (I), can lead to better accumulation of the compound at the receptor sites targeted by the group Y of the compound. Without wishing to be bound by theory, the present inventors believe that the presence of the albumin-binding group facilitates increased binding of compounds of Formula (I) to the desired receptor site through the group Y of the compound by slowing the elimination of the compound from the circulation after administration. Since the time over which the compounds persist in the circulation is increased (i.e. increased residence time due to reduced renal filtration and excretion), the overall effect is that compounds of Formula (I) show binding and uptake through the desired receptor site. When compared to analogous compounds without the albumin-binding group, the binding of the compounds of the present invention is increased. Where a greater proportion of the administered dose of the compound is able to bind to the desired receptor site, this may allow for a smaller dose or amount to be administered, which improves overall efficacy. The presence of an albumin-binding group may also permit administration of a single dose of the compound, rather than repeat dosing (i.e. administration of multiple doses) to ensure sufficient binding of the compound at the target site for the purposes of imaging or therapy.

The present inventors have found that groups of Formula (II) and subformulae, show the ability to bind to serum albumin that is present in the blood of a subject. In some embodiments, Z is a group capable of binding to serum albumin. The compounds of Formula (I) where Z is a group of Formula (II) therefore show greater binding to the targeted tumor sites in vivo, since the compounds bind to albumin and persist in the circulation for longer. This then allows for more opportunity and time for the compound of Formula (I) to bind to the desired receptor through the group Y of the compound.

In some embodiments, Z is an albumin-binding group of Formula (II):

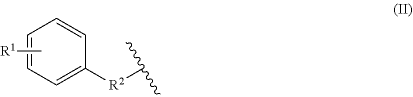

wherein:
R¹ is an optionally substituted C₁-C₁₂ alkyl group, optionally substituted C₁-C₂₀ alkoxy group, halogen or CN group; and
R² is an optionally substituted C₂-C₁₆ alkylene group, wherein one or more alkylene units in the alkylene group may be replaced with a group selected from a urea, thiourea, amine, amide, carbonyl heteroatom or arylene group, wherein the arylene group may be optionally substituted.

In a preferred embodiment, R¹ is a C₁ alkyl or C₁ alkoxy group. In another preferred embodiment, R¹ is halogen group, particularly I or Cl. In another preferred embodiment, R¹ is a CN group.

In other embodiments, R² is an optionally substituted C₂-C₁₆ alkylene group. In another embodiment, R² is an optionally substituted C₂-C₁₀ alkylene group. In other embodiments, one alkylene group of R² is replaced with a urea, thiourea, amine, amide, carbonyl or heteroatom group. In another embodiment, R² is a C₁-C₆ alkylene group optionally substituted by a carboxylic acid group.

In some embodiments, the group Z of Formula (III) has one of the following structures:

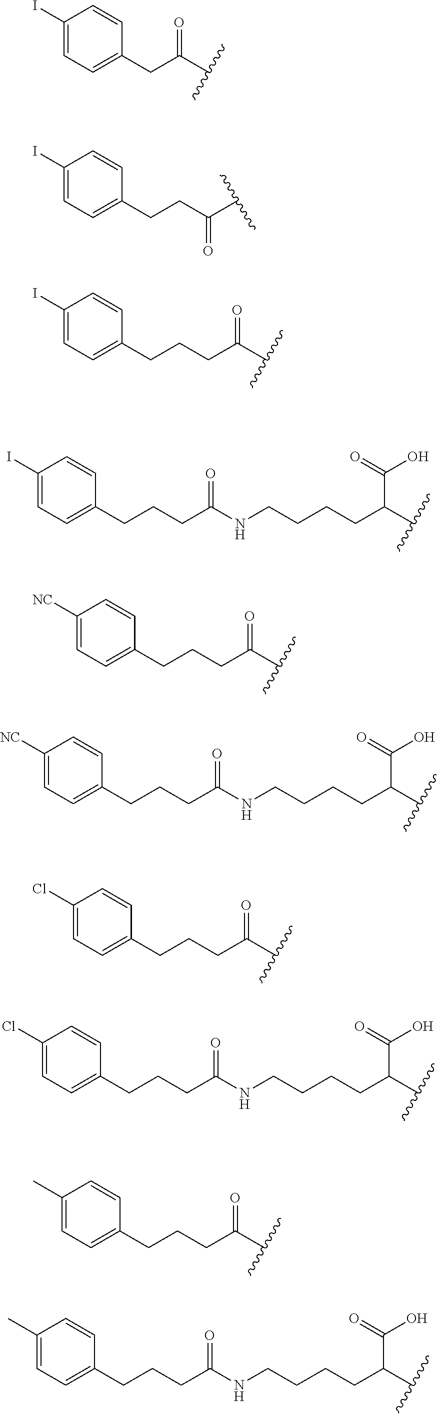

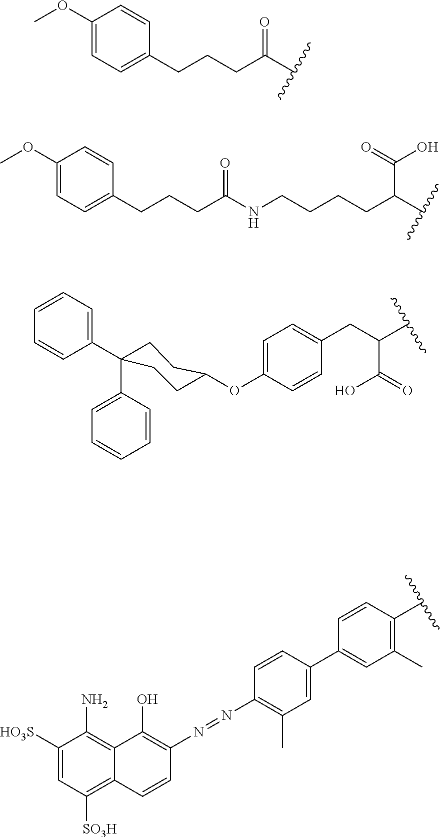 [SMILES: *C(=O)CCCc1ccc(OC)cc1 ; *C(CCCCNC(=O)CCCc1ccc(OC)cc1)C(=O)O ; *C(Cc1ccc(OC2CCC(c3ccccc3)(c3ccccc3)CC2)cc1)C(=O)O ; *c1ccc(-c2ccc(N=Nc3ccc4c(S(=O)(=O)O)cc(S(=O)(=O)O)c(N)c4c3O)c(C)c2)cc1C]

In an embodiment, Z is a derivative of iodophenylbutyric acid. In another embodiment, Z has the following structure:

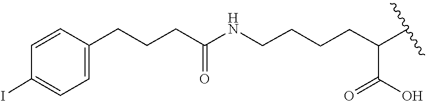 [SMILES: *C(CCCCNC(=O)CCCc1ccc(I)cc1)C(=O)O]

As used herein, the term “optionally substituted” denotes that the group may or may not be further substituted or fused (so as to form a condensed polycyclic system), with one or more non-hydrogen substituent groups. In certain embodiments the substituent groups are one or more groups independently selected from the group consisting of halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalkylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkyloxy, alkyloxyalkyl, alkyloxycycloalkyl, alkyloxyheterocycloalkyl, alkyloxyaryl, alkyloxyheteroaryl, alkyloxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, —C(═O)OH, —C(═O)R^{a}, —C(═O)OR^{a}, C(═O)NR^{a}R^{b}, C(═NOH)R^{a}, C(═NR^{a})NR^{b}R^{c}, NR^{a}R^{b}, NR^{a}C(═O)R^{b}, NR^{a}C(═O)OR^{b}, NR^{a}C(═O)NR^{b}R^{c}, NR^{a}C(═NR^{b})NR^{c}R^{d}, NR^{a}SO₂R^{b}, —SR^{a}, SO₂NR^{a}R^{b}, —OR^{a}, OC(═O)NR^{a}R^{b}, OC(═O)R^{a} and acyl, wherein R^{a}, R^{b}, R^{c} and R^{d} are each independently selected from the group consisting of H, C₁-C₁₂alkyl, C₁-C₁₂haloalkyl, C₂-C₁₂alkenyl, C₂-C₁₂alkynyl, C₂-C₁₀ heteroalkyl, C₃-C₁₂cycloalkyl, C₃-C₁₂cycloalkenyl, C₂-C₁₂heterocycloalkyl, C₂-C₁₂ heterocycloalkenyl, C₆-C₁₈ aryl, C₁-C₁₈heteroaryl, and acyl, or any two or more of R^{a}, R^{b}, R^{c} and R^{d}, when taken together with the atoms to which they are attached form a heterocyclic ring system with 3 to 12 ring atoms.

In some embodiments, each optional substituent is independently selected from the group consisting of: halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, hydroxy, hydroxyalkyl, alkyloxy, alkyloxyalkyl, alkyloxyaryl, alkyloxyheteroaryl, alkenyloxy, alkynyloxy, cycloalkyloxy, cycloalkenyloxy, heterocycloalkyloxy, heterocycloalkenyloxy, aryloxy, heteroaryloxy, arylalkyl, heteroarylalkyl, arylalkyloxy, amino, alkylamino, acylamino, aminoalkyl, arylamino, sulfonyl, alkylsulfonyl, arylsulfonyl, aminosulfonyl, aminoalkyl, —COOH, —SH, and acyl.

Examples of particularly suitable optional substituents include F, Cl, Br, I, CH₃, CH₂CH₃, OH, OCH₃, CF₃, OCF₃, NO₂, NH₂, COOH, COOCH₃ and CN.

As used herein, the term “alkyl” refers to a group or part of a group that is a straight or branched aliphatic hydrocarbon group, preferably a C₁-C₁₂ alkyl, more preferably a C₁-C₁₀ alkyl, most preferably C₁-C₆ unless otherwise noted. Examples of suitable straight and branched C₁-C₆ alkyl substituents include methyl, ethyl, n-propyl, 2-propyl, n-butyl, sec-butyl, t-butyl, hexyl, and the like.

As used herein, the term “halogen” represents chlorine, fluorine, bromine or iodine.

As used herein, the term “heteroatom” refers to a nitrogen (N), oxygen (O) or sulfur (S) atom.

As used herein, the term “alkylene” refers to a bivalent straight or branched chain aliphatic hydrocarbon group. For example, a C₂-C₁₆ alkylene group is a bivalent hydrocarbon group with 2 to 16 carbon atoms in the chain.

As used herein, the term “arylene” refers to a bivalent cyclic aromatic group. For example, a C₅-C₁₂ arylene group is a bivalent cyclic aromatic group with C₅-C₁₂ carbon atoms.

As used herein, the term “alkoxy” refers to a group that has an alkyl group bound to an oxygen atom. Examples of alkoxy groups include methoxy (MeO—), ethoxy (EtO—), propoxy (PrO) and aryloxy (ArO—), where the aryl group is a cyclic aromatic group.

As used herein, the terms “urea” and “thiourea” refer to a —NH—C(O)—NH— and a —NH—C(S)—NH— functional group, respectively. The urea and thiourea groups are divalent in nature and can replace one or more alkylene groups in an alkylene chain. Since the urea and thiourea functional groups are symmetrical, the orientation (i.e. forward or reverse) of the groups result in the same structure.

As used herein, the term “amine” refers to a —NH₂ or —NH— group, where the valency of the group depends on the surrounding atoms. For example, where an amine group replaces an alkylene unit, the amine group will be an —NH— group. Where the amine group is in a terminal position, the amine group will be a —NH₂ group. One or more hydrogen atoms (where appropriate) may be replaced with non-hydrogen groups, which will result in a substituted amine.

As used herein, the term “amide” refers to a —NH—C(O)— group. It will be understood that the amide group may be present in either the forward or reverse directions and the reference to an amide group encompasses both versions.

The compounds of the present invention contain a group capable of recognizing and subsequently binding to a biological receptor, i.e. the group Y in the compound of Formula (I). In an embodiment, Y is a protein, a fragment thereof or a derivative thereof. In another embodiment, Y is a peptide, a fragment thereof or a derivative thereof. In an embodiment, Y is a polypeptide, a fragment thereof or a derivative thereof. In an embodiment, Y is a carbohydrate, a fragment thereof or a derivative thereof. In an embodiment, Y is an oligonucleotide, a fragment thereof or a derivative thereof. In an embodiment, Y is a liposome, a fragment thereof or a derivative thereof. In an embodiment, Y is an oligosaccharide, a fragment thereof or a derivative thereof. In an embodiment, Y is an antibody, a fragment thereof or a derivative thereof. In an embodiment, Y is a steroid, a fragment thereof or a derivative thereof. In an embodiment, Y is a nucleic acid, a fragment thereof or a derivative thereof. In an embodiment, Y is folic acid, a fragment thereof or a derivative thereof. In an embodiment, Y is vitamin B12, a fragment thereof or a derivative thereof. The group Y in compounds of Formula (I) may be selected on the basis of its ability to bind specifically to a particular receptor site in vivo, where the receptor is a known marker or indicator for a given disease or indication.

In some embodiments, Y is selected from the group consisting of octreotate, octreotide, [Tyr³]-octreotate, bombesin, bombesin(7-14), peptides binding to prostate specific membrane antigen (PSMA), gastrin releasing peptide, penetratin, annexin V, TAT peptide, cyclic RGD, glucose, glucosamine, folic acid, neurotensin, neuropeptide Y, cholecystokinin (CCK) analogues, vasoactive intestinal peptide (VIP), substance P and alpha-melanocyte-stimulating hormone (MSH).

In an embodiment, Y is octreotate. In another embodiment, Y is a bombesin. In another embodiment, Y is bombesin(7-14). In another embodiment, Y is a peptide that binds to PSMA.

In another embodiment, Y is a cyclic RGD. In another embodiment, Y is the fragment cRGDfK.

In an embodiment, Y is octreotate and has one of the following structures:

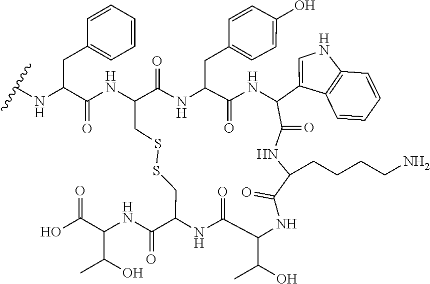 [SMILES: *NC(Cc1ccccc1)C(=O)NC1CSSCC(C(=O)NC(C(=O)O)C(C)O)NC(=O)C(C(C)O)NC(=O)C(CCCCN)NC(=O)C(c2c[nH]c3ccccc23)NC(=O)C(Cc2ccc(O)cc2)NC1=O]

In another embodiment, Y is octreotate with the following stereochemistry:

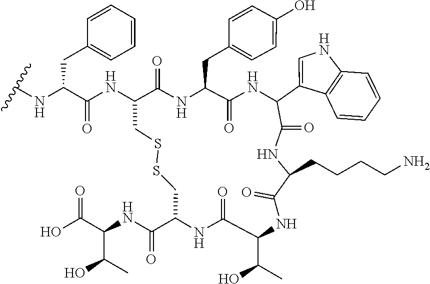 [SMILES: *N[C@H](Cc1ccccc1)C(=O)N[C@H]1CSSC[C@@H](C(=O)N[C@H](C(=O)O)[C@@H](C)O)NC(=O)[C@H]([C@@H](C)O)NC(=O)[C@H](CCCCN)NC(=O)C(c2c[nH]c3ccccc23)NC(=O)[C@H](Cc2ccc(O)cc2)NC1=O]

In an embodiment, Y is a bombesin. In an embodiment, Y is a bombesin having an amino acid sequence of D-Phe-Gln-Trp-Ala-Val-Gly-His-Sta-Leu-NH₂. In another embodiment, Y is a bombesin with one of the following structures:

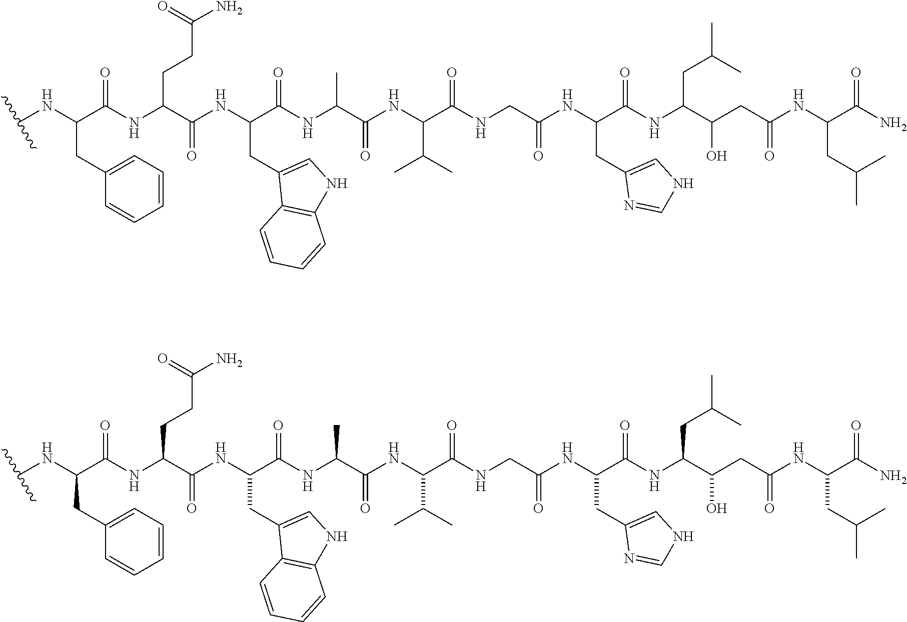 [SMILES: *NC(Cc1ccccc1)C(=O)NC(CCC(N)=O)C(=O)NC(Cc1c[nH]c2ccccc12)C(=O)NC(C)C(=O)NC(C(=O)NCC(=O)NC(Cc1c[nH]cn1)C(=O)NC(CC(C)C)C(O)CC(=O)NC(CC(C)C)C(N)=O)C(C)C ; *N[C@H](Cc1ccccc1)C(=O)N[C@@H](CCC(N)=O)C(=O)N[C@@H](Cc1c[nH]c2ccccc12)C(=O)N[C@@H](C)C(=O)N[C@H](C(=O)NCC(=O)N[C@@H](Cc1c[nH]cn1)C(=O)N[C@@H](CC(C)C)[C@@H](O)CC(=O)N[C@@H](CC(C)C)C(N)=O)C(C)C]

In an embodiment, Y is a peptide that binds to PSMA. In another embodiment, Y is a peptide that binds PSMA and has one of the following structures:

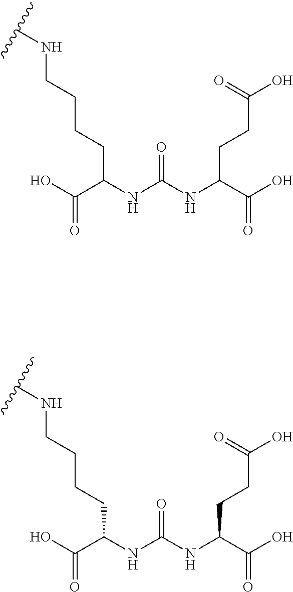 [SMILES: *NCCCCC(NC(=O)NC(CCC(=O)O)C(=O)O)C(=O)O ; *NCCCC[C@H](NC(=O)N[C@@H](CCC(=O)O)C(=O)O)C(=O)O]

In another embodiment, Y is a cyclic RGD. In another embodiment, Y is the fragment RGDfK. In another embodiment, Y has the following structure:

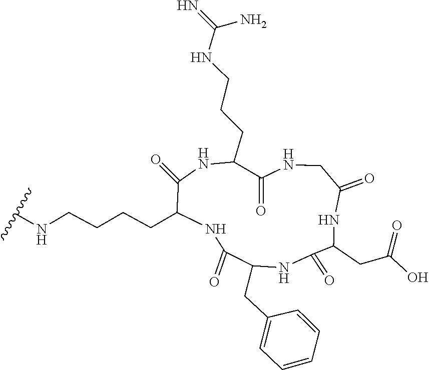 [SMILES: *NCCCCC1NC(=O)C(Cc2ccccc2)NC(=O)C(CC(=O)O)NC(=O)CNC(=O)C(CCCNC(=N)N)NC1=O]

In another embodiment, Y has the following stereochemistry:

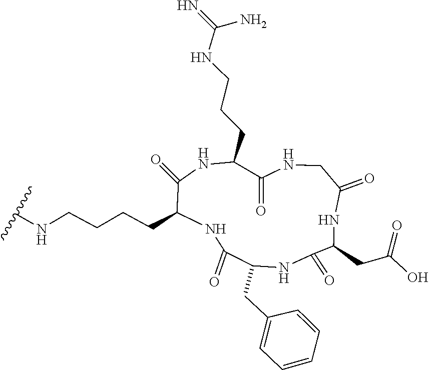

The compounds of Formula (I) contain a sarcophagine and a group capable of binding to a biological receptor, where the latter is bound to terminal position of the sarcophagine via a linker group. As depicted herein, the linker group comprises a propylamide group bound directly to the terminal position of the sarcophagine. The propylamide group is then attached to further linker group comprising a polyethylene oxide group, having between 1 and 10 repeat units. The polyethylene oxide group has the following structure:

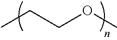

where n is an integer from 1 to 10.

In an embodiment, n is 1. In another embodiment, n is 2. In another embodiment, n is 3. In another embodiment, n is 4. In another embodiment, n is 5. In another embodiment, n is 6. In another embodiment, n is 7. In another embodiment, n is 8. In another embodiment, n is 9. In another embodiment, n is 10.

In an embodiment, the compound of Formula (I) has one of the following structures, which represent specific compounds of Formula (I) where n is 3 or 4:

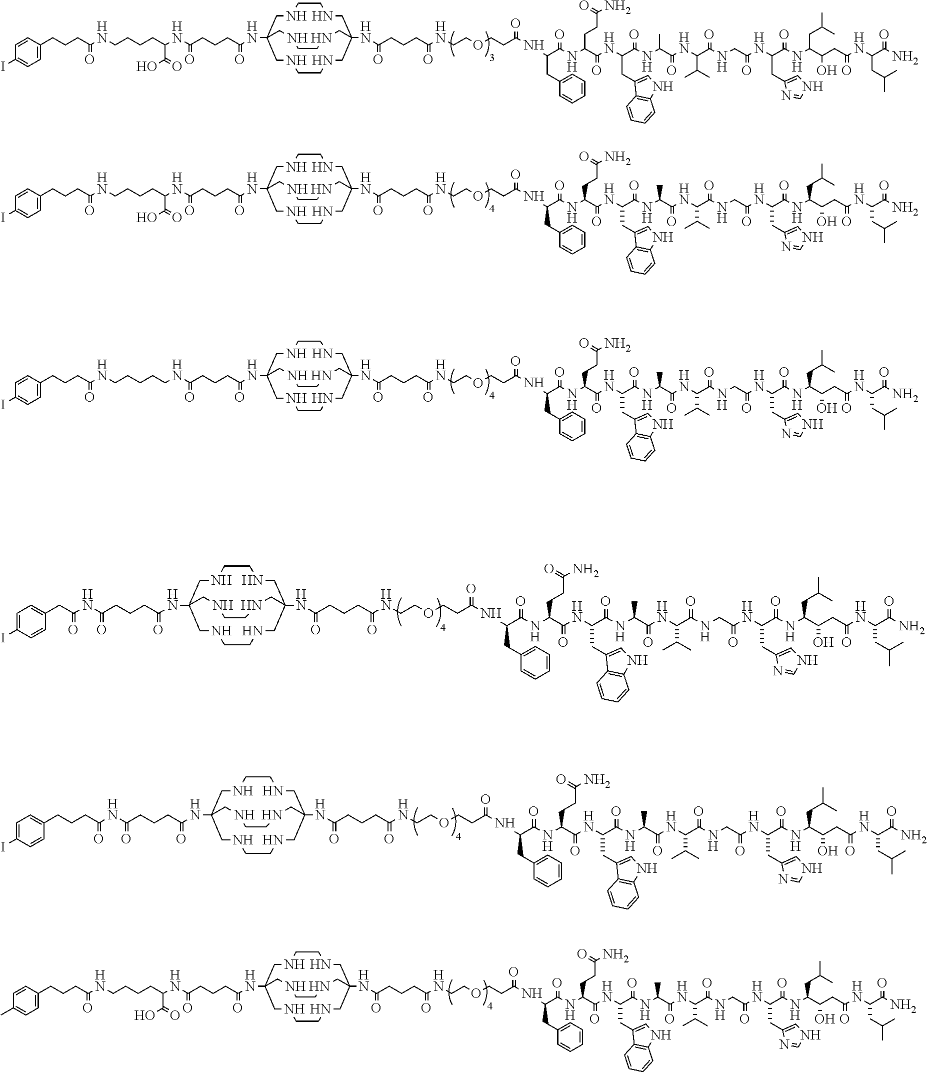

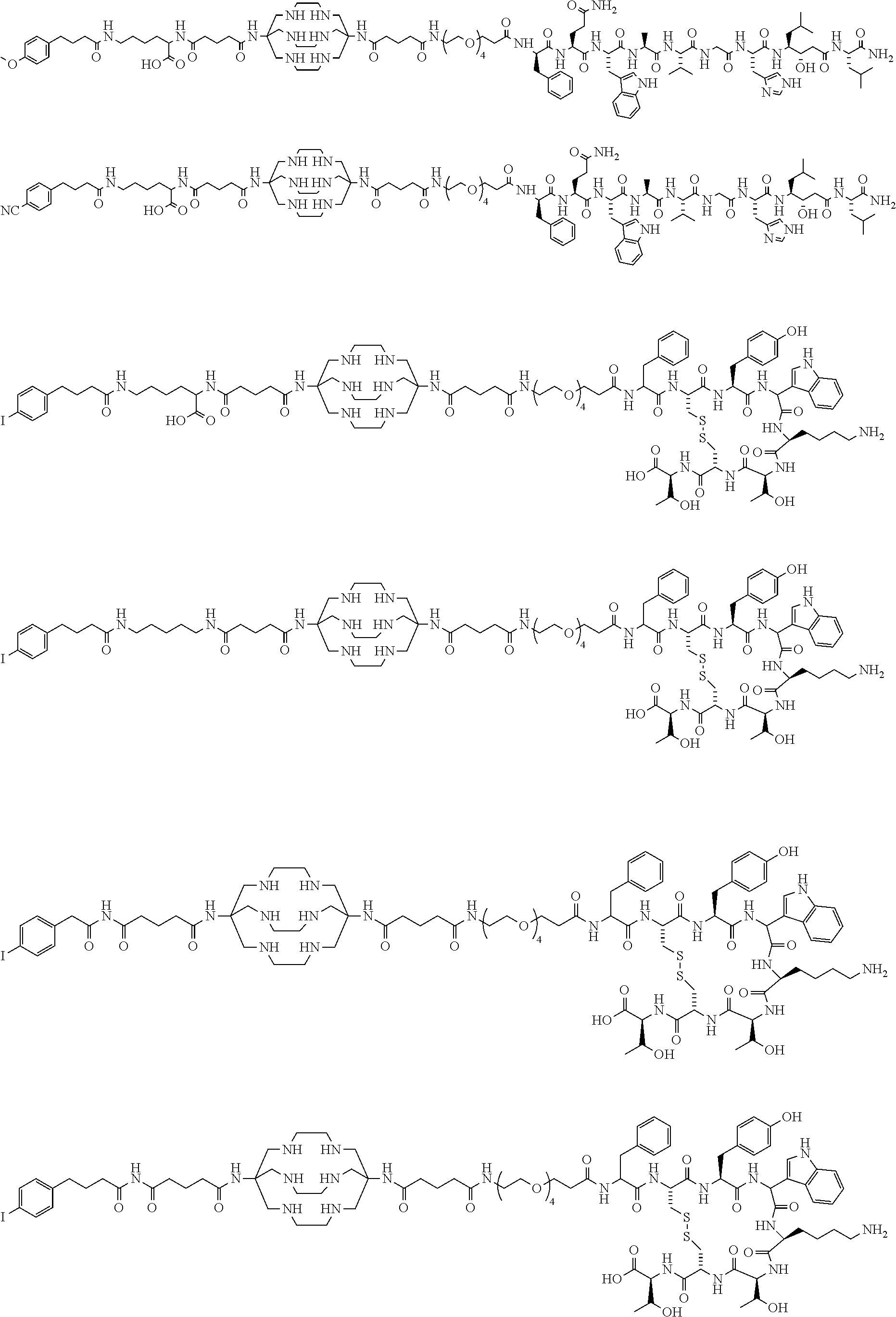

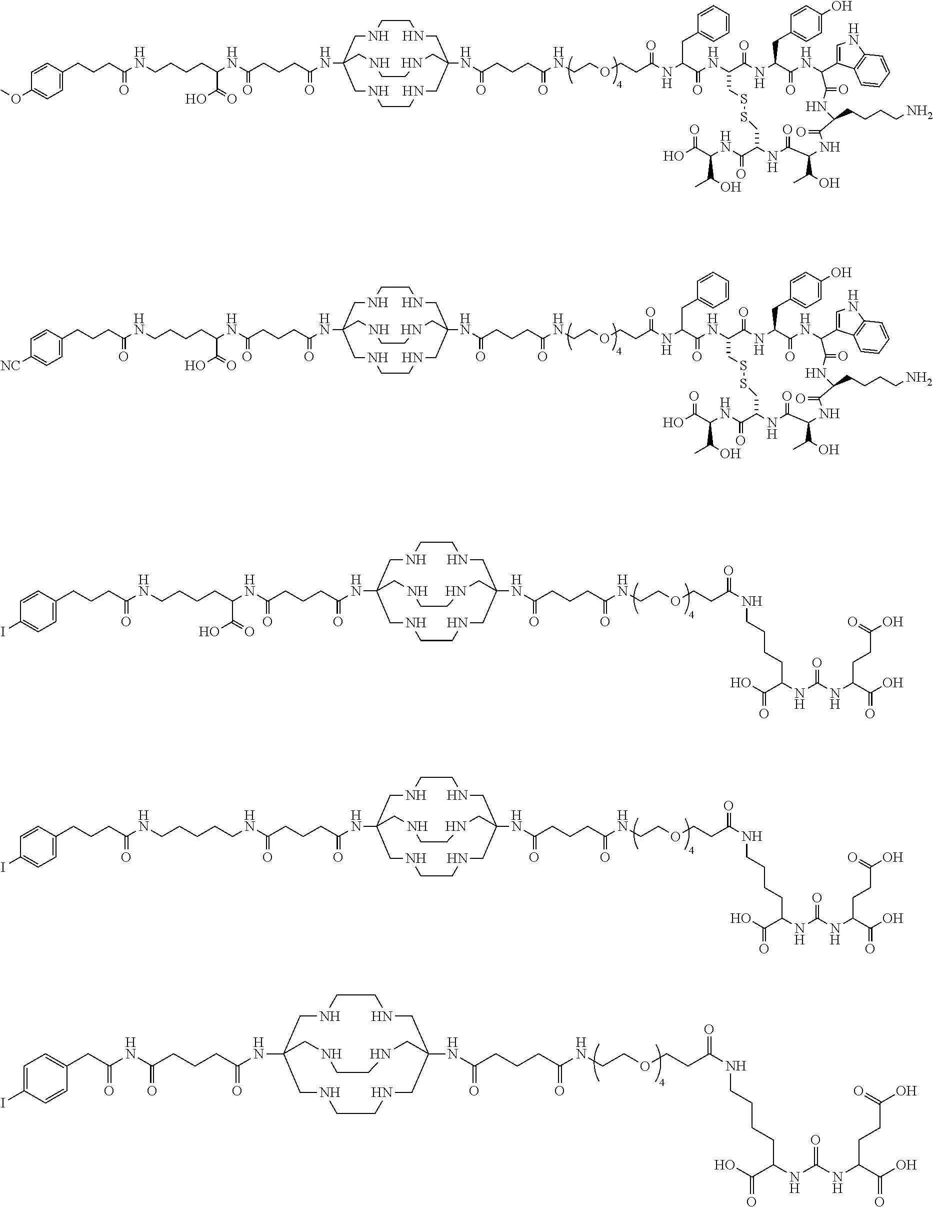

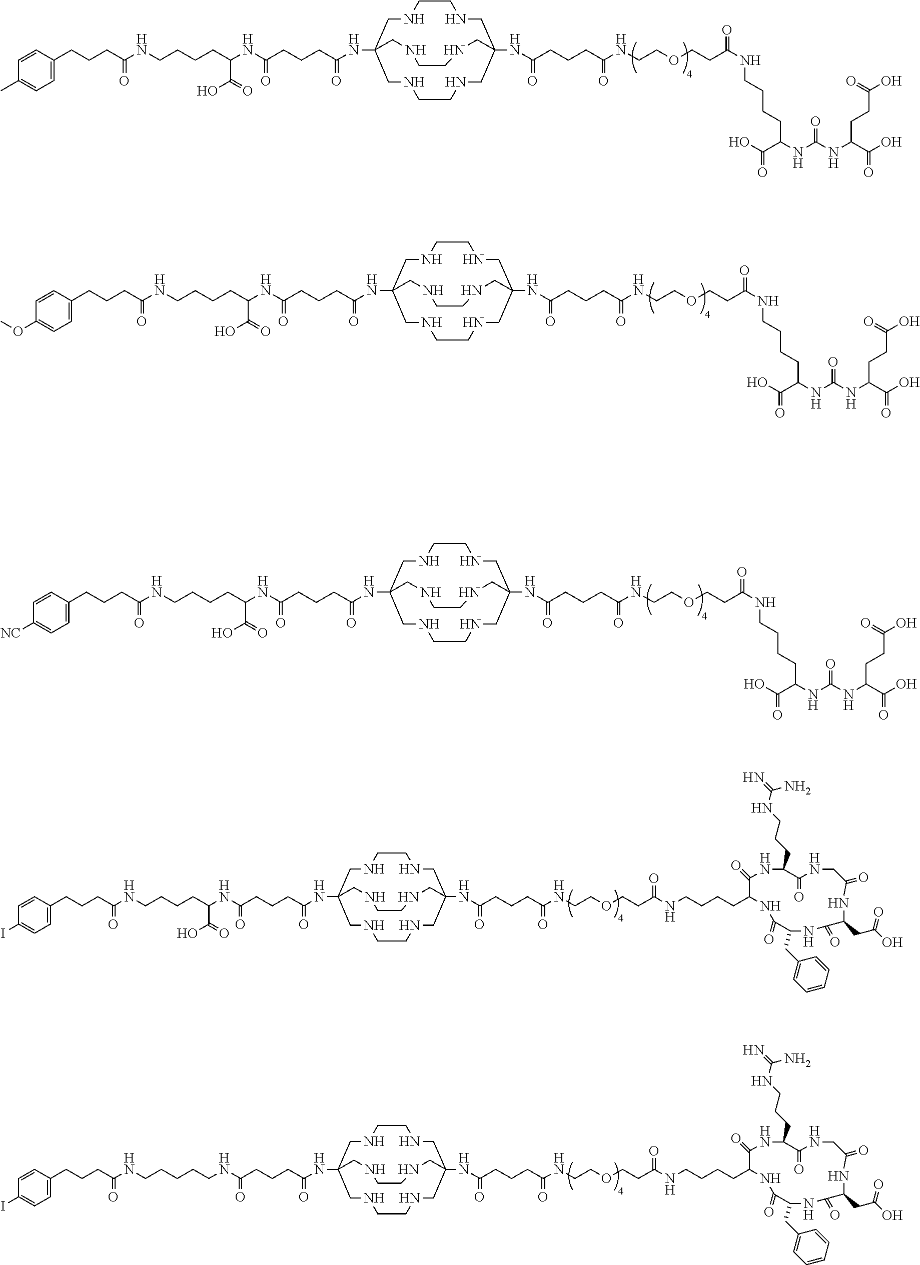

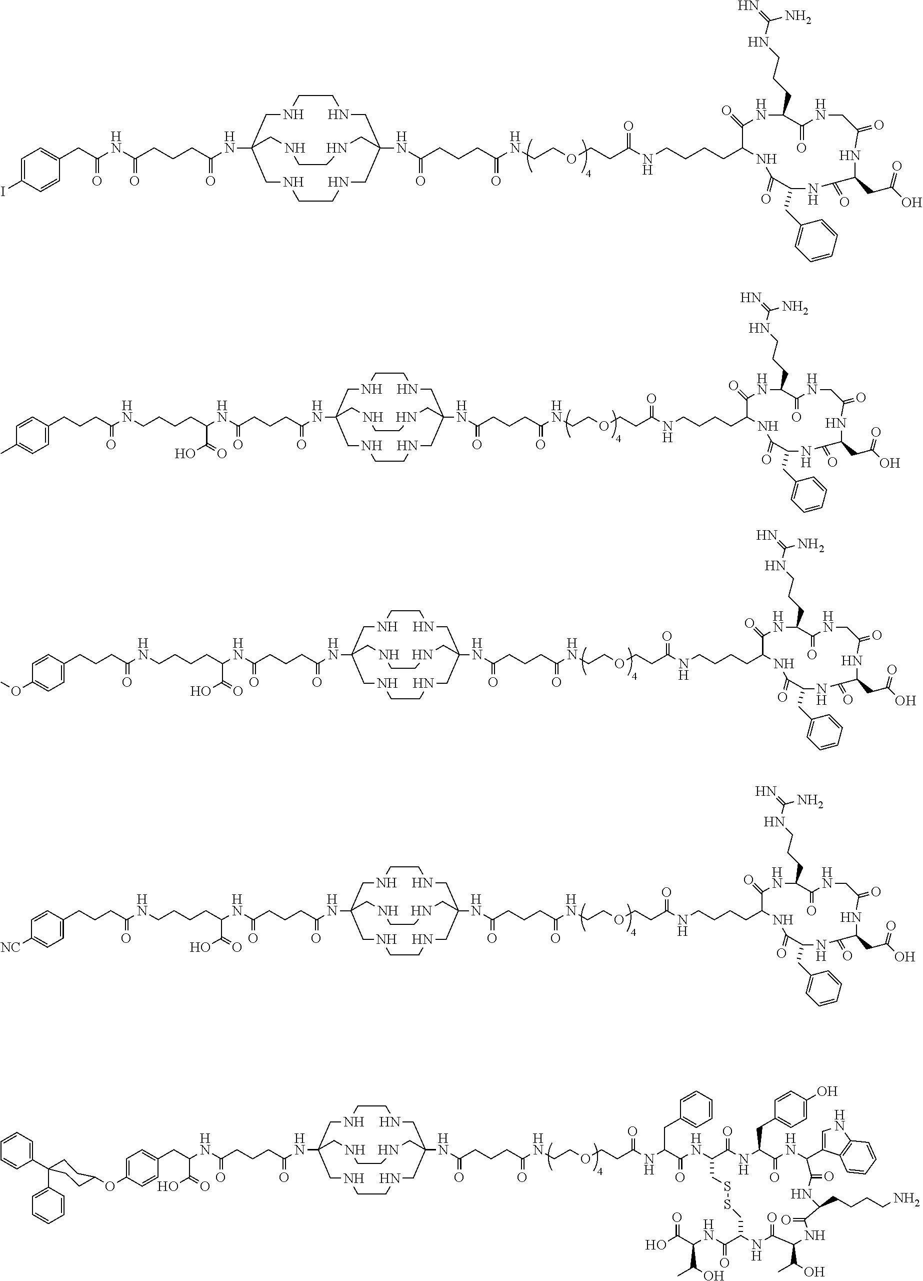

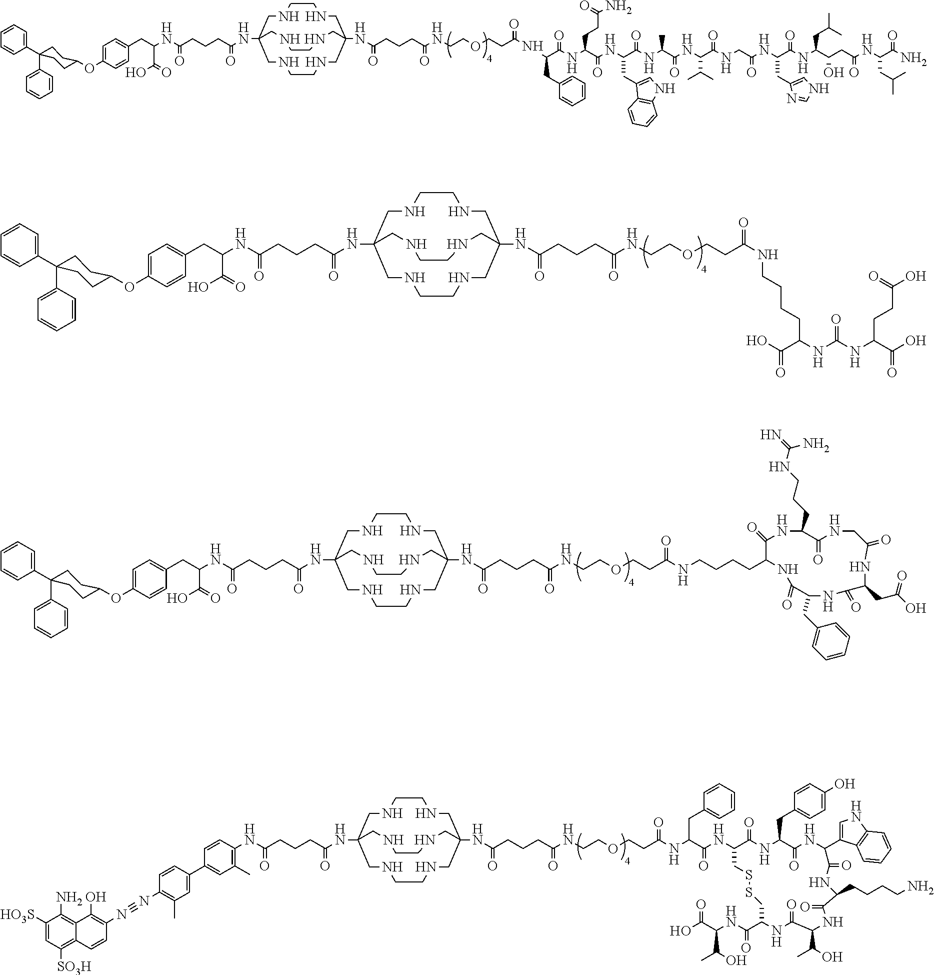

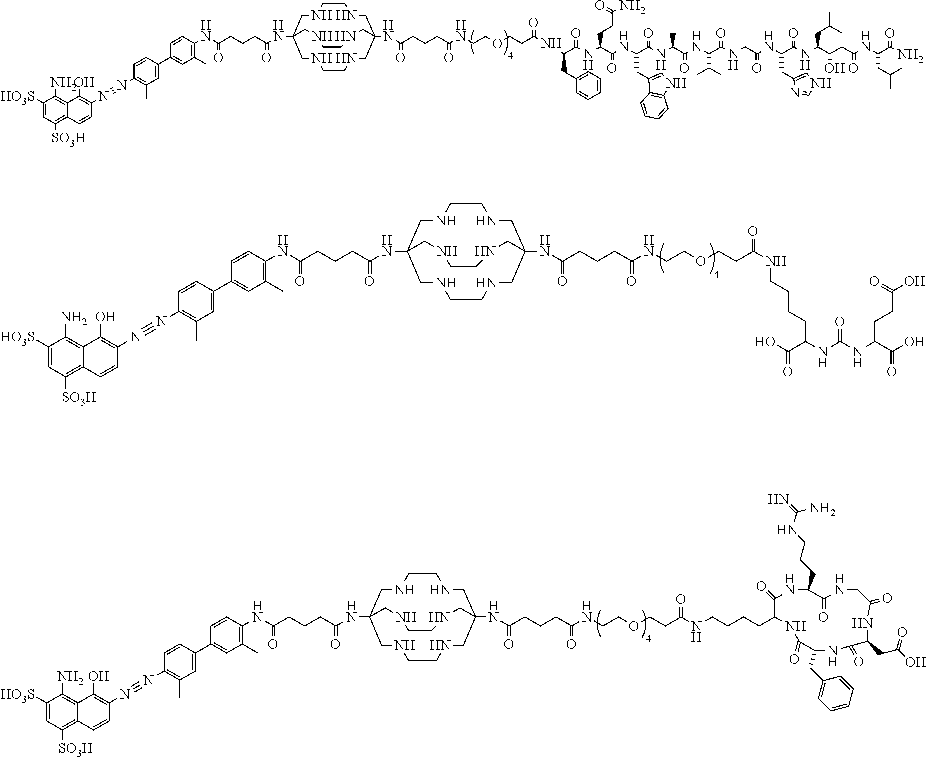

The compounds of Formula (I) may be coordinated with a metal ion via the nitrogen-containing macrocycle to form the corresponding complexes of Formula (I). In an embodiment, the compound of Formula (I) is coordinated with a metal ion.

In an embodiment, the metal ion is an ion of Cu, Tc, Gd, Ga, In, Co, Re, Fe, Au, Mg, Ca, Ag, Rh, Pt, Bi, Cr, W, Ni, V, Ir, Zn, Cd, Mn, Ru, Pd, Hg, Ti, Lu, Sc, Zr, Pb, Ac and Y.

In an embodiment, the metal ion is a radionuclide. In some embodiments, the metal ion is a radionuclide of a metal selected from the group consisting of Cu, Tc, Ga, Co, In, Fe, and Ti. The present compounds have been found to be particularly useful in binding copper ions.

In some embodiments, the metal ion is a radionuclide selected from the group consisting of ⁶⁰Cu, ⁶¹Cu, ⁶²Cu, ⁶⁴Cu and ⁶⁷Cu. In some embodiments the radionuclide is ⁶⁰Cu. In some embodiments the radionuclide is ⁶¹Cu. In some embodiments the radionuclide is ⁶²Cu. In some embodiments the radionuclide is ⁶⁴Cu. In some embodiments the radionuclide is ⁶⁷Cu. The compounds of the present invention have also been useful in binding cobalt ions. In some embodiments, the metal ion is a radionuclide of ⁵⁵Co. In some embodiments the radionuclide is ⁵⁷Co. In some embodiments the radionuclide is 58mCoThe compounds of the present invention have also been useful in binding indium ions. In some embodiments, the metal ion is ¹¹¹In. The compounds of the present invention may also prove useful in binding scandium ions. In some embodiments the metal ion is ⁴³Sc. In some embodiments the metal ion is ⁴⁴Sc. In some embodiments the metal ion is ⁴⁷Sc.

Where the metal ion is a radionuclide and the compound for Formula (I) is radiolabelled to form a complex, the complex may be administered for the purposes of radiotherapy or radioimaging. As discussed earlier, compounds (and subsequently, the radiolabelled complexes) of Formula (I) contain a group Y that is capable of binding a biological receptor, therefore the radiolabelled complexes of Formula (I) may be used for the radiotherapy or radioimaging of cancers that are associated with overexpression of the receptor to which the group Y may bind.

The present inventors have found that the compounds and complexes of Formula (I) containing a sarcophagine, a group capable of binding a biological receptor, an albumin binding group, the propylamide linker and the linker comprising a PEG group shows affinity for the biological receptor. The combination of each of these components in the compound of Formula (I) allow for administration of the corresponding complex containing a radionuclide, maintaining stability of the complex in vivo and accumulation of the complex at the intended target. The compounds of the present invention contain both an albumin-binding group and a group capable of binding a biological receptor. In order for both of these groups to bind to their respective targets, there must be a sufficient distance between them in order to prevent any reaction between the groups. The present inventors have found that the combination of the linker groups (i.e. the propylamide linker and the linker comprising the PEG group) and the sarcophagine itself provides a compound where the distance between the albumin-binding group and the group capable of binding the biological receptor prevents any such reaction. In addition to contributing to the requisite distance between the albumin-binding group and the group capable of binding a biological receptor, the linker comprising the PEG group modifies the lipophilicity of the compound, which in turn improves the hydrolytic stability of the compound and its various fragments.

The compounds of the present invention and complexes thereof with a radionuclide may be used in methods of radioimaging, diagnosis or treatment. In some embodiments, the compounds of the present invention complexed with a radionuclide may be used in a method for radioimaging, diagnosis or treatment of a cancer.

As used herein the terms “treating”, “treatment”, “preventing”, “prevention” and grammatical equivalents refer to any and all uses which remedy the stated neuroendocrine tumour, prevent, retard or delay the establishment of the disease, or otherwise prevent, hinder, retard, or reverse the progression of the disease. Thus the terms “treating” and “preventing” and the like are to be considered in their broadest context. For example, treatment does not necessarily imply that a patient is treated until total recovery. Where the disease displays or a characterized by multiple symptoms, the treatment or prevention need not necessarily remedy, prevent, hinder, retard, or reverse all of said symptoms, but may prevent, hinder, retard, or reverse one or more of said symptoms.

As used herein, the term “cancer” broadly encompasses neoplastic diseases characterised by abnormal cell growth with the potential to invade or spread to other parts of the body. The cancer may be benign, which does not spread to other parts of the body. The cancer may be malignant, meaning that the cancer cells can spread through the circulatory system or lymphatic system. The term as used herein includes all malignant, i.e. cancerous, disease states. The cancer may be present as a tumour. Accordingly, the term “tumour” is used generally to define any malignant cancerous or pre-cancerous cell growth, and may include leukemias, but is particularly directed to solid tumours or carcinomas such as melanomas, colon, lung, ovarian, skin, breast, pancreas, pharynx, brain, prostate, CNS, and renal cancers (as well as other cancers).

As used herein, the term “tumour” refers to any malignant cancerous or pre-cancerous cell growths. The term may also include leukemias, but is particularly directed to solid tumours or carcinomas.

Radioimaging of a cancer of associated with the expression of a receptor in connection with the administration of a complex of Formula (I) also relies upon the selection of a suitable radionuclide. For example, where the intended use of a complex of Formula (I) is for the purposes of radioimaging, the selected radionuclide should have a sufficiently long half-life such that detection of radionuclide decay allows for images of a sufficient quality to be obtained. This also requires that the compound of Formula (I) itself, i.e. the ligand coordinating the radionuclide, be sufficiently stable with respect to radioactive decay. The present inventors have found that decomposition of a complex of Formula (I) by radiolysis (i.e. as a result of the radioactivity of the radionuclide) is minimized and that the complex of Formula (I) generally remains intact in this regard.

Radioimaging of a subject to which a radiolabeled compound of Formula (I) is administered may be by positron emission tomography (PET) or by single-photon emission computed tomography (SPECT). In an embodiment, the present invention provides a method for radioimaging a subject in need thereof, the method comprising administering a compound of Formula (I) complexed with a radionuclide. In an embodiment, the method comprises administering a compound of Formula (I) complexed with a copper radionuclide. In another embodiment, the method comprises administering a compound of Formula (I) complexed with ⁶⁴CU.

In an embodiment, radioimaging of the subject after administration of the compound of Formula (I) complexed by a radionuclide is by PET. In another embodiment, radioimaging of the subject after administration of the compound of Formula (I) complexed by a radionuclide is by SPECT.

The compounds of the present invention complexed with a radionuclide may be administered to a subject in need thereof as a composition by a parenteral route. Administration by intravenous injection may be preferred. Alternatively, the formulations of the present invention may be given by intraarterial or other routes, for delivery into the systemic circulation. The subject to which the compound is administered is then placed into a PET (or SPECT) scanner and images showing the localisation of the complex, and subsequently location of any cancers or tumours, are obtained. This then allows for diagnosis and detection of a cancer or tumour.

The term “subject” as used herein refers to mammals and includes humans, primates, livestock animals (e.g. sheep, pigs, cattle, horses, donkeys), laboratory test animals (e.g. mice, rabbits, rats, guinea pigs), performance and show animals (e.g. horses, livestock, dogs, cats), companion animals (e.g. dogs, cats) and captive wild animals. Preferably, the mammal is human or a laboratory test animal. Even more preferably, the mammal is a human.

The compounds of the present invention and complexes thereof with a radionuclide may be used in methods of treatment of diseases, such as cancers. When complexed with a suitable radionuclide, the complexes of the present invention may be administered to a subject in need thereof. The methods disclosed herein comprise administration of a therapeutically effective amount of a radiolabeled compound of the present invention to a subject in need thereof. In an embodiment, the present invention provides a method for treating a disease in a subject in need thereof, the method comprising administering a therapeutically effective amount of compound of Formula (I) complexed with a radionuclide.

The term “therapeutically effective amount” or “effective amount” is an amount sufficient to effect beneficial or desired clinical results. An effective amount can be administered in one or more administrations. For the purposes of radioimaging, an effective amount is sufficient for an image showing the localisation of the compound of Formula (I) administered to the subject, owing to the detection of the products of decay from the radioisotope that is complexed with the compound. For the purposes of treatment, an effective amount is typically sufficient to palliate, ameliorate, stabilize, reverse, slow and/or delay the progression of the cancer.

In an embodiment, the method comprises administering a compound of Formula (I) complexed with a copper radionuclide. In another embodiment, the method comprises administering a compound of Formula (I) complexed with ⁶⁷Cu. In an embodiment, the method comprises administering a compound of Formula (I), which comprises an octreotate group. In another embodiment, the method comprises administering a compound of Formula (I), which comprises a bombesin group. In another embodiment, the method comprises administering a compound of Formula (I), which comprises a peptide that binds to PSMA. In another embodiment, the method comprises administering a compound of Formula (I), which comprises a cyclic RGD group.

The selection of a particular group represented by Y in the compound of Formula (I) is based on a receptor-ligand interaction that may be attributed to a particular cancer or indication where the expression of the receptor is increased as a result of the cancer. Given the abundance of such receptors are associated with a particular type of cancer, the accumulation of compounds of the present invention as detected by the radioactive decay of the radionuclide indicates the location of the cancer. The present inventors have found that compounds of the present invention show a particular affinity for the receptor to be targeted. Furthermore, the presence of both the propylamide linker and the linker comprising PEG group contribute to provide a complex (when the compound is radiolabeled with a radionuclide) that is capable of administration to a subject and subsequent localization at sites overexpressing the receptor that is targeted. The compounds of the present invention also have the requisite stability with respect to the radionuclide. For example, the sarcophagine present in the compound is capable of chelating a radionuclide such that the radionuclide remains coordinated upon administration to a subject and subsequent binding at the target site. Since the radionuclide remains coordinated and localized to the target site due to binding of the compound as a whole, radiation damage at other sites (e.g. healthy tissue) is minimized.

In an embodiment, the present invention provides a method for treating a cancer, the method comprising administering a compound of Formula (I) complexed with a radionuclide. In an embodiment, the method comprises administering a compound of Formula (I) complexed with a copper radionuclide. In another embodiment, the method comprises administering a compound of Formula (I) complexed with ⁶⁷Cu. In an embodiment, the method comprises administering a compound of Formula (I), which comprises an octreotate group. In another embodiment, the method comprises administering a compound of Formula (I), which comprises a bombesin group. In another embodiment, the method comprises administering a compound of Formula (I), which comprises a peptide that binds to PSMA. In another embodiment, the method comprises administering a compound of Formula (I), which comprises a cyclic RGD group.

The compounds and complexes of the present invention can be administered alone or in the form of a pharmaceutical composition in combination with a pharmaceutically acceptable carrier, diluent or excipient. The compounds of the invention, while effective themselves, are typically formulated and administered in the form of their pharmaceutically acceptable salts as these forms are typically more stable, more easily crystallised and have increased solubility.

The compounds are, however, typically used in the form of pharmaceutical compositions which are formulated depending on the desired mode of administration. The compositions are prepared in manners well known in the art.

In using the compounds of the invention they can be administered in any form or mode which makes the compound available for the desired application (imaging or radiotherapy). One skilled in the art of preparing formulations of this type can readily select the proper form and mode of administration depending upon the particular characteristics of the compound selected, the condition to be treated, the stage of the condition to be treated and other relevant circumstances. Reference is made to Remington's Pharmaceutical Sciences, 19th edition, Mack Publishing Co. (1995) for further information.

The invention in other embodiments provides a pharmaceutical pack or kit comprising one or more containers filled with one or more of the ingredients of the pharmaceutical compositions of the invention. In such a pack or kit can be found at least one container having a unit dosage of the agent(s). Conveniently, in the kits, single dosages can be provided in sterile vials so that the clinician can employ the vials directly, where the vials will have the desired amount and concentration of compound and radio nucleotide which may be admixed prior to use. Associated with such container(s) can be various written materials such as instructions for use, or a notice in the form prescribed by a governmental agency regulating the manufacture, use or sale of pharmaceuticals, imaging agents or biological products, which notice reflects approval by the agency of manufacture, use or sale for human administration.

In an embodiment, the invention provides compositions comprising a compound as described above together with one or more pharmaceutically acceptable excipients.

Pharmaceutical compositions of this invention for parenteral injection comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin.

If desired, and for more effective distribution, the compounds can be incorporated into slow release or targeted delivery systems such as polymer matrices, liposomes, and microspheres.

The injectable formulations can be sterilized, for example, by filtration through a bacterial-retaining filter, or by incorporating sterilizing agents in the form of sterile solid compositions that can be dissolved or dispersed in sterile water or other sterile injectable medium just prior to use.

The present invention also provides processes for the synthesis or preparation of compounds of the invention. The present inventors have found that established procedures that may be used for the preparation of compounds of the present invention by various coupling procedures and conditions does not allow the desired compounds to be accessed. This is largely due to incompatibilities between functional groups, issues of reagent solubility and general issues of reactivity. For example, in Scheme 1 below, while compounds of Formula (I) may be produced, the overall yield of the compound is low. The present inventors have found that the manner and the order in which the groups Y and Z are attached to the corresponding linker groups affects the outcome of the reaction to install the group on the compound and also the yield of the reaction. In Scheme 1, group Y is installed first, however this leads to loss of the oxygen protecting group (e.g. the hydroxysuccinimide group) at which the group Z is then attached thus leading to lower yields overall. The present inventors have also found that in some cases, the group Y is also modified during the coupling reaction, which is undesirable since the ability of the compound as a whole to bind to the desired target site depends on the group Y being intact.

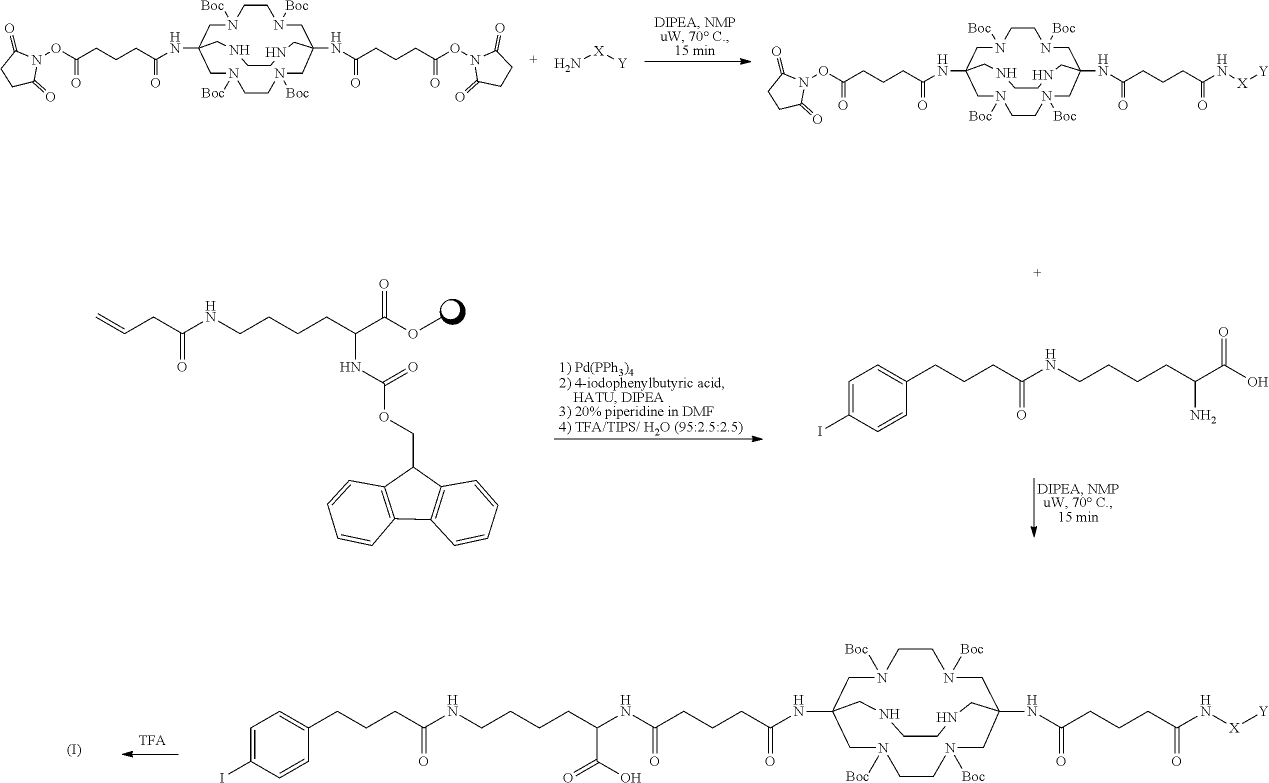

The present inventors have now found that compounds of the present invention may be accessed according to the process described in Scheme 2 below.

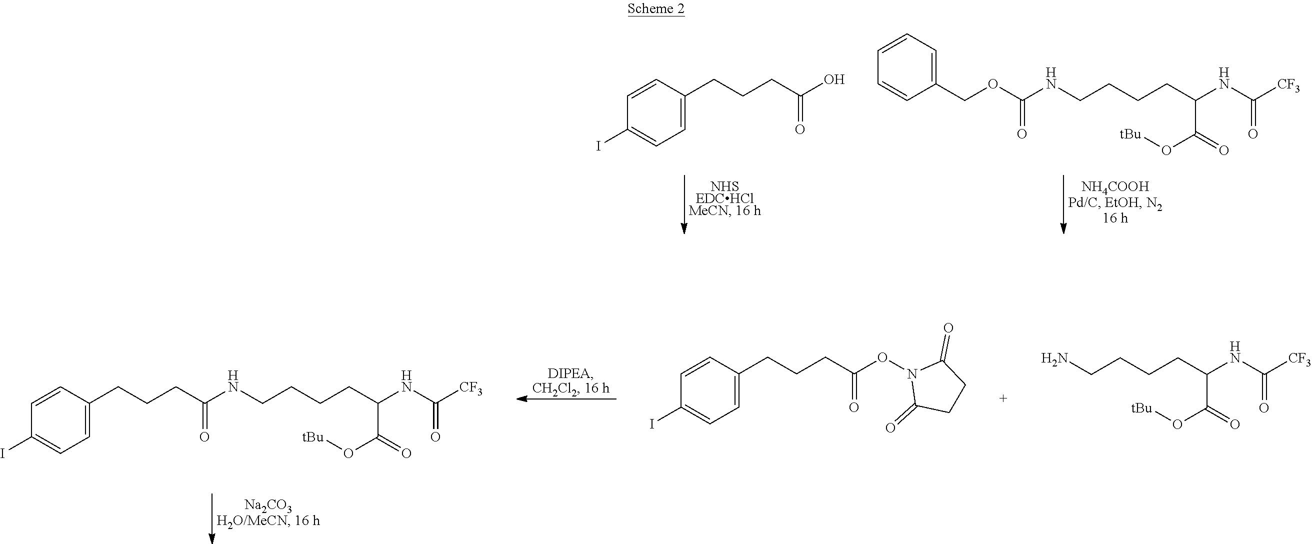

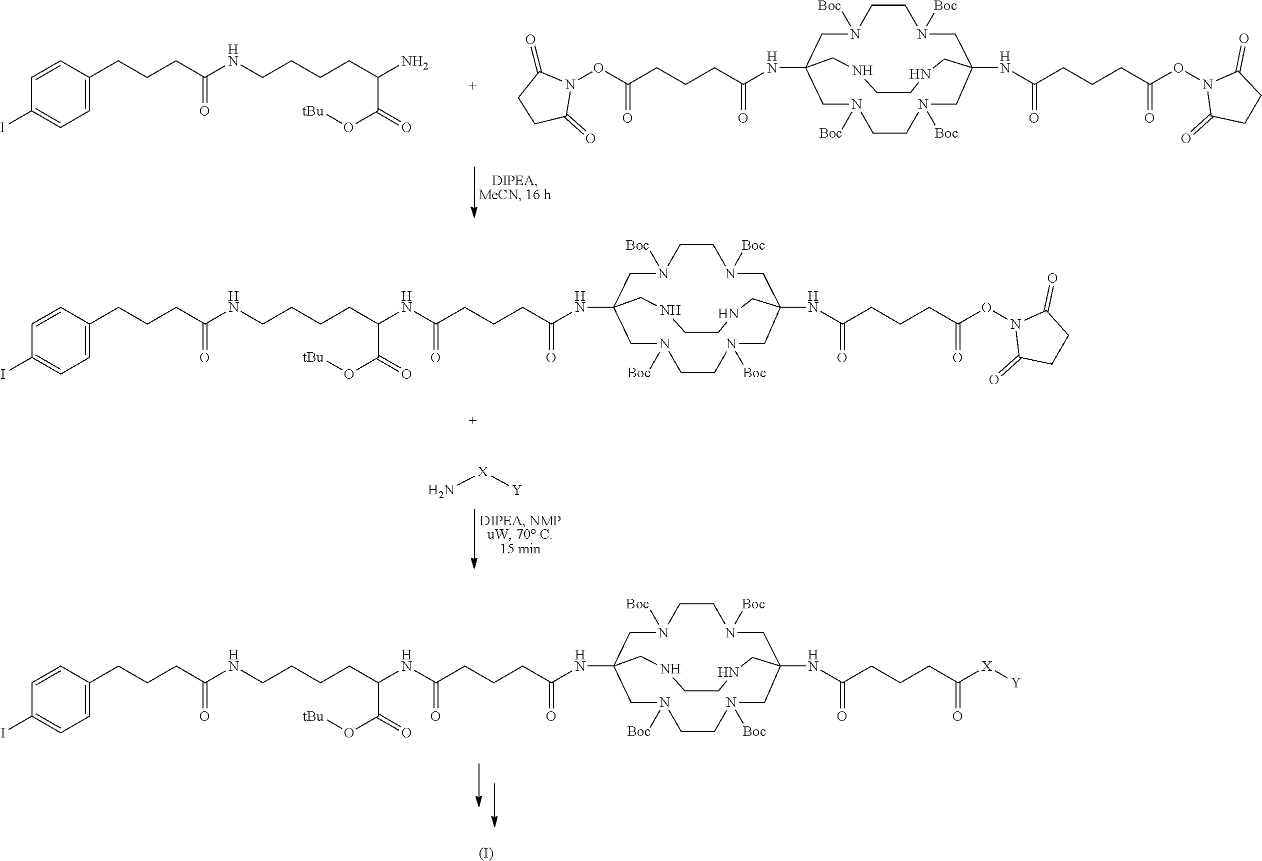

According to an aspect of the present invention, there is provided a process for producing a compound of Formula (I) or a salt, complex, isomer, solvate or protected form thereof:

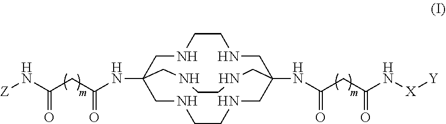

wherein m, X, Y and Z are as defined in an earlier aspect, the method comprising the steps of:
i) coupling a compound of Formula (IV), or a salt, complex, isomer or solvate thereof,

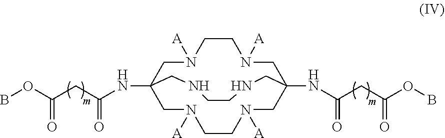

with a compound of Formula (V) or a salt thereof,

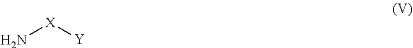

for a time and under conditions to give a compound of Formula (VI) or a salt thereof

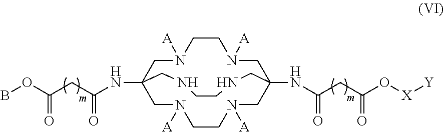

wherein A is a nitrogen-protecting group and B is an oxygen-protecting group;
ii) coupling a compound of Formula (VI) of step i) with a compound of Formula (VII) or a salt thereof:

H₂N—Z (VII)
for a time and under conditions to give a compound of Formula (VIII) or a salt thereof

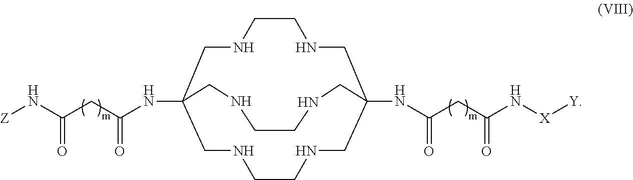

The present inventors have found that the process defined in Scheme 2 allows for a more efficient synthesis of compounds of Formula (I). As described above, a key step in the process involves a sarcophagine bearing appropriate protecting groups that allow for the subsequent coupling reactions to occur at the intended sites only. The processes disclosed herein comprise two coupling reactions to install a group that is capable of binding to a biological receptor (i.e. Y in a compound of Formula (I)) and an albumin-binding group (i.e. Z in a compound of Formula (I)). Various coupling conditions are possible, with the suitable coupling conditions dependent on the nature of the coupling partners to be coupled to the sarcophagine. The conditions may be modified by a person skilled in the art. Various protecting groups are also possible, with the requirement that the compounds will tolerate the protection and deprotection conditions. A person skilled in the art will understand that the choice of the protecting groups will depend on the reaction conditions required for their installation and removal. The protected form of the compound (i.e. the compound of Formula (VIII)) obtained as a process as described herein can then be deprotected by the appropriate means to arrive at the compounds of Formula (I). As the steps to couple the compounds of Formulae (IV) and (V) and Formulae (VI) and (VII) result in the formation of one or more amide (i.e. peptide) bonds, there may be multiple approaches that will be suitable, for example, solution phase peptide coupling or solid phase peptide conditions may be used.

In an embodiment, the processes defined in the present invention produce a compound of Formula (Ia) or a salt, complex, isomer, solvate or protected form thereof:

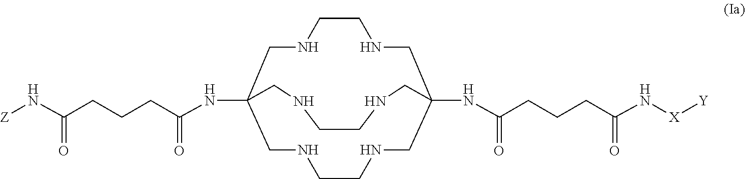

wherein X, Y and Z are as defined in the second aspect, the method comprising the steps of:
i) coupling a compound of Formula (IVa), or a salt, complex, isomer or solvate thereof,

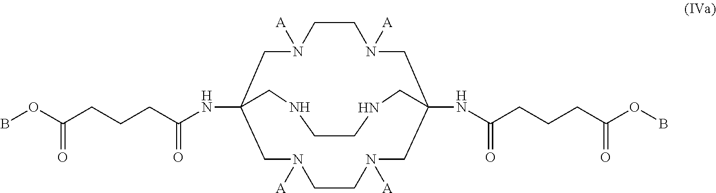

with a compound of Formula (Va) or a salt thereof,

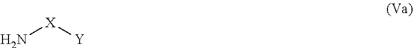

for a time and under conditions to give a compound of Formula (VIa) or a salt thereof

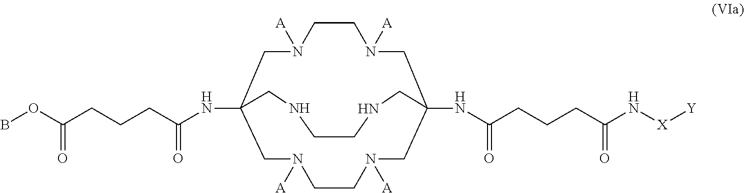

wherein A is a nitrogen-protecting group and B is an oxygen-protecting group;
ii) coupling a compound of Formula (VIa) of step i) with a compound of Formula (VIIa) or a salt thereof:

H₂N—Z (VIIa)
for a time and under conditions to give a compound of Formula (VIIIa) or a salt thereof

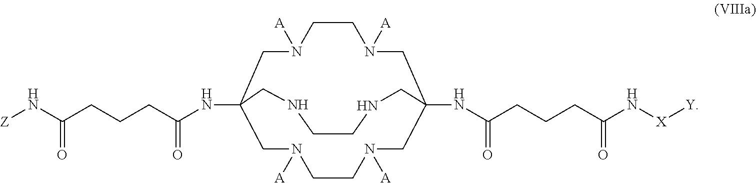

As used herein, the term “oxygen protecting group” refers to a group that can prevent the oxygen moiety reacting during further derivatisation of the protected compound and which can be readily removed when desired. In one embodiment the protecting group is removable in the physiological state by natural metabolic processes. Examples of oxygen protecting groups include acyl groups (such as acetyl), esters (such as methyl, t-butyl and benzyl esters) ethers (such as methoxy methyl ether (MOM), β-methoxy ethoxy methyl ether (MEM), p-methoxy benzyl ether (PMB), methylthio methyl ether, pivaloyl (Piv), tetrahydropyran (THP)), silyl ethers, such as trimethylsilyl (TMS) tert-butyl dimethyl silyl (TBDMS) and triisopropylsilyl (TIPS) and succinimides, such as N-hydroxysuccinimide.

As used herein, the term “nitrogen protecting group” refers to a group that can prevent the nitrogen moiety reacting during further derivatisation of the protected compound and which can be readily removed when desired. In one embodiment the protecting group is removable in the physiological state by natural metabolic processes and in essence the protected compound is acting as a prodrug for the active unprotected species. Examples of suitable nitrogen protecting groups that may be used include formyl, trityl, phthalimido, acetyl, trifluoroacetyl, trichloroacetyl, chloroacetyl, bromoacetyl, iodoacetyl; urethane-type blocking groups such as benzyloxycarbonyl (‘CBz’), 4-phenylbenzyloxycarbonyl, 2-methylbenzyloxycarbonyl, 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, t-butoxycarbonyl (“tBoc”), 2-(4-xenyl)-isopropoxycarbonyl, 1,1-diphenyleth-1-yloxycarbonyl, 1,1-diphenylprop-1-yloxycarbonyl, 2-phenylprop-2-yloxycarbonyl, 2-(p-toluyl)-prop-2-yloxy-carbonyl, cyclo-pentanyloxy-carbonyl, 1-methylcyclopentanyloxycarbonyl, cyclohexanyloxycarbonyl, 1-methylcyclohexanyloxycarbonyl, 2-methylcyclohexanyloxycarbonyl, 2-(4-toluylsulfono)-ethoxycarbonyl, 2-(methylsulfono)ethoxycarbonyl, 2-(triphenylphosphino)-ethoxycarbonyl, fluorenylmethoxycarbonyl (“Fmoc”), 2-(trimethylsilyl)ethoxycarbonyl, allyloxycarbonyl, 1-(trimethylsilylmethyl)prop-1-enyloxycarbonyl, 5-benzisoxalymethoxy carbonyl, 4-acetoxybenzyloxycarbonyl, 2,2,2-trichloroethoxycarbonyl, 2-ethynyl-2-propoxycarbonyl, cyclopropylmethoxycarbonyl, 4-(decycloxy)benzyloxycarbonyl, isobornyloxycarbonyl, 1-piperidyloxycarbonlyl and the like; benzoylmethylsulfono group, 2-nitrophenylsulfenyl, diphenylphosphine oxide, and the like. The actual nitrogen protecting group employed is not critical so long as the derivatised nitrogen group is stable to the condition of subsequent reaction(s) and can be selectively removed as required without substantially disrupting the remainder of the molecule including any other nitrogen protecting group(s). Further examples of these groups are found in: Greene, T. W. and Wuts, P. G. M., Protective Groups in Organic Synthesis, Second edition; Wiley-Interscience: 1991; Chapter 7; McOmie, J. F. W. (ed.), Protective Groups in Organic Chemistry, Plenum Press, 1973; and Kocienski, P. J., Protecting Groups, Second Edition, Thieme Medical Pub., 2000.

In an embodiment, the oxygen protecting group is a succinimide. In another embodiment, the oxygen protecting group is an N-hydroxysuccinimide. In another embodiment, the oxygen protecting group is a tert-butoxycarbonyl group. In another embodiment, the oxygen protecting group is a tert-butyl ester.

In an embodiment, the nitrogen protecting group is an acetyl group. In another embodiment, the nitrogen protecting group is a trifluoroacetyl group. In another embodiment, the nitrogen protecting group is a tert-butoxycarbonyl group.

In certain embodiments, step i) of the process is performed under microwave conditions. In certain embodiments, step ii) of the process is performed under microwave conditions. In certain embodiments, steps i) and ii) of the process is performed under microwave conditions. The present inventors have found that the use of microwave conditions allows access to compounds of Formula (I) in better yields. Without wishing to be bound by theory, the present inventors believe that performing the requisite coupling reactions under microwave conditions allows for fewer unwanted side products to be obtained, which also simplifies any purification and isolation procedures required.

In an embodiment, the process for producing a compound of Formula (I) further comprises the step of reacting the compound of Formula (I) with a metal ion for a time and under conditions such that a complex of Formula (I) and the metal ion is formed. In an embodiment, the metal ion is a copper ion. In another embodiment, the copper ion is a radionuclide. In another embodiment, the copper ion is selected from the group consisting of ⁶⁰Cu, ⁶¹Cu, ⁶²Cu, ⁶⁴Cu and ⁶⁷Cu.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### Examples

The following examples are illustrative of the disclosure and should not be construed as limiting in any way the general nature of the disclosure of the description throughout this specification.

### Synthesis of Compounds of the Invention

The agents of the various embodiments may be prepared using the reaction routes and synthesis schemes as described below, employing the techniques available in the art using starting materials that are readily available. The preparation of particular compounds of the embodiments is described in detail in the following examples, but the artisan will recognize that the chemical reactions described may be readily adapted to prepare a number of other agents of the various embodiments. For example, the synthesis of non-exemplified compounds may be successfully performed by modifications apparent to those skilled in the art, e.g. by appropriately protecting interfering groups, by changing to other suitable reagents known in the art, or by making routine modifications of reaction conditions. A list of suitable protecting groups in organic synthesis can be found in T.W. Greene's Protective Groups in Organic Synthesis, 3^{rd} Edition, John Wiley & Sons, 1991. Alternatively, other reactions disclosed herein or known in the art will be recognized as having applicability for preparing other compounds of the various embodiments. Reagents useful for synthesizing compounds may be obtained or prepared according to techniques known in the art.

### Instrumentation

Mass spectra were collected using a Thermo Scientific Exactive Plus OrbiTrap LC/MS (Thermo Fisher Scientific, Massachusetts, USA) and calibrated to internal references.

NMR spectra were recorded on an Agilent MR400 NMR (California, USA) (¹H at 400 MHz) at 297 K and referenced in internal solvent residue.

Analytical RP-HPLC traces were recorded using an Agilent 1200 HPLC system equipped with an Alltech Hypersil BDS C18 analytical HPLC column (4.6×150 mm, 5 μm) with a flow rate of 1 mL min⁻¹, with UV absorbance being recorded at 214 and 254 nm. Retention times (Rₜ/min) were recorded using a gradient elution of 5-100% B in A (A=0.1% TFA, B=MeCN with 0.1% TFA) over 30 min.

Semi-preparative HPLC was performed on an Agilent 1200 HPLC System using a buffer of A=0.1% TFA and B=0.1% TFA in MeCN with UV detection at 214 nm.

Microwave synthesis was performed using a Biotage (Uppsala, Sweden) Initator+microwave system.

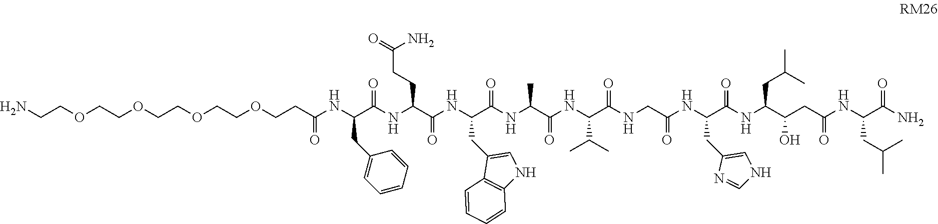 [SMILES: CC(C)C[C@H](NC(=O)C[C@H](O)[C@H](CC(C)C)NC(=O)[C@H](Cc1c[nH]cn1)NC(=O)CNC(=O)[C@@H](NC(=O)[C@H](C)NC(=O)[C@H](Cc1c[nH]c2ccccc12)NC(=O)[C@H](CCC(N)=O)NC(=O)[C@@H](Cc1ccccc1)NC(=O)CCOCCOCCOCCOCCN)C(C)C)C(N)=O]

The peptide was synthesised using an automated, microwave assisted peptide synthesiser (Liberty Blue, CEM, NC, USA) using standard Fmoc-SPPS techniques utilising HATU and DIPEA as coupling reagents on a Rink Amide solid support (125 mg, 0.8 mmol/g, 0.1 mmol. The crude peptide was cleaved from the solid support using TFA/TIPS/H₂O (95:2.5:2.5) before being evaporated to dryness under a stream of N₂ gas. The resulting residue was then dissolved in 30% MeCN in water and purified by semi-preperative HPLC (30% to 100% B in A over 70 min, Phenomonex Luna C18, λ=254 nm). Fractions containing the desired product were collated and lyophilised to yield a colourless powder (26 mg, 0.002 mmol, 2% yield) OTOF/MS [C₆₆H₁₀₁N₁₅O₁₆+2H]²⁺ m/z 680.886 (experimental), 680.885 (calc'd); [C₆₆H₁₀₁N₁₅O₁₆+3H]³⁺ m/z 454.259 (experimental), 454.259 (calc'd). RP-HPLC (λ=254 nm) Rt=11.0 min.

(tBoc)₄₋₅sar(NHS)₂

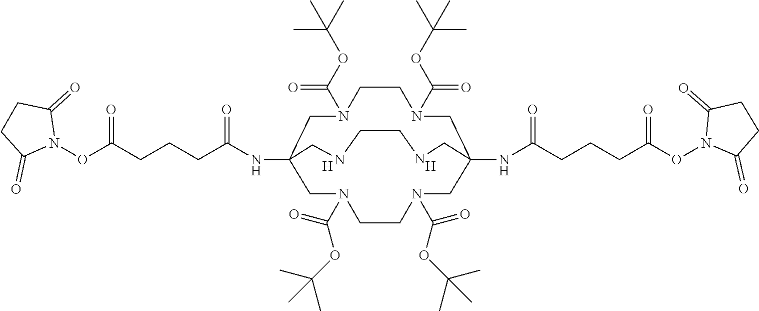

A solution of sar(CO₂H)₂·xHCl (446 mg, 0.82 mmol if x=0) in H₂O/MeCN (1:1, 30 mL) was treated with tBoc₂O (1.02 g, 4.67 mmol) and DIPEA (1 mL, 5.74 mmol) and stirred for 16 h at room temperature. The solvent was then removed under reduced pressure and the resultant residue was suspended in MeCN (40 mL) and treated with EDC·HCl (501 mg, 2.61 mmol) and N-hydroxysuccinamide (300 mg, 2.61), resulting in the dissolution of the suspension. The solution was then evaporated to dryness under reduced pressure and the resulting residue was extracted with CHCl₃ (80 mL), washed with water (3×40 mL) followed by brine (40 mL). The organic extract was then dried over Na₂SO₄, filtered, and evaporated to dryness under reduced pressure to yield a pink-hued residue. The residue was purified by column chromatography (SiO₂, 70 mL, 5% MeOH in CH₂Cl₂, Rf=0.54) to yield a mixture of both (tBu)₄sar(NHS)₂ and (tBu)₅sar(NHS)₂ (136 mg). OTOF/MS [C₅₂H₈₄N₁₀O₁₈+H]⁺ m/z 1137.60 (experimental), 1137.60 (calc'd); [C₅₇H₉₂N₁₀O₂₀+H]⁺ m/z 1237.66 (experimental), 1237.66 (calc'd).

p-Iodophenylbutyryl-lysine (H-Lys(IPB)-OH)

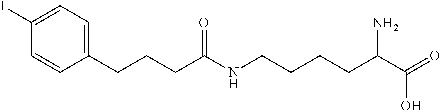 [SMILES: NC(CCCCNC(=O)CCCc1ccc(I)cc1)C(=O)O]

Fmoc-(Alloc)Lys-OH (1.60 g, 3.56 mmol) was activated with HBTU (1.33 g, 3.51 mmol), DIPEA (1.2 mL, 6.90 mmol) and DMAP (54 mg, 0.44 mmol) for 30 min before being applied to Wang resin (2.65 g, 1.15 mmoleq/g, 3.04 mmol) and allowed to incubate for 1 h. The resin was then capped by the addition for acetic anhydride (0.6 mL 5.44 mmol) and pyridine (0.5 mL, 6.44 mmol) which was allowed to incubate for 1 h before the loaded resin was isolated by filtration. The resin was then treated with a mixture of Pd(PPh₃)₄ (380 mg, 0.33 mmol) and morpholine (0.53 mL, 6.13 mmol) in dichloromethane (10 mL) for 3 h. The resin was filtered and the removal of the alloc-group was confirmed by MS analysis. A mixture of p-Iodophenylbutyric acid (1.03 g, 3.55 mmol), HATU (1.28 g, 3.36 mmol) and DIPEA (1.3 mL, 7.29 mmol) in DMF (20 mL) was incubated with shaking for 30 min before being applied to the lysine-loaded resin and allowed to react for 16 h at ambient temperature. The resin was then filtered, washed with DMF then dichloromethane and air dried. The Fmoc-group was then removed by washing with piperazine in DMF (20%, 15 mL) for 15 min and repeated three times.

The resin was then treated with TFA:H₂O:TIPS (95:2.5:2.5, 8 mL) for 1 h. The resin was then filtered and the filtrate concentrated. Pentane was then added, precipitating an off-white solid that was collected, washed with pentane and air dried to yield an off-white powder (461 mg, 1.11 mmol, 31% yield based on resin loading).

TFA-Lys(Z)-(OtBu)

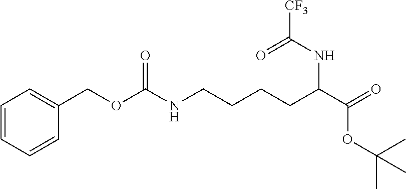 [SMILES: CC(C)(C)OC(=O)C(CCCCNC(=O)OCc1ccccc1)NC(=O)C(F)(F)F]

A solution of H-Lys(Z)-OtBu-HCl (3.16 g, 8.47 mmol) in methanol (50 mL) was treated with DIPEA (3 mL) followed by ethyl trifluoroacetate (1.5 mL, 12.6 mmol). The mixture was stirred at room temperature for 16 h. The solvent was then evaporated to dryness under reduced pressure to yield a colourless oil which was extracted into ethyl acetate (50 mL), washed with HCl (0.01 M, 50 mL) followed by water (2×50 mL). The organic fraction was then dried over MgSO₄, filtered and concentrated. The addition of pentane resulted in the precipitation of a colourless, crystalline solid that was isolated by filtration and air dried (2.50 g, 5.78 mmol, 68% yield). ¹H NMR (400 MHz; CDCl₃): δ_{H}/ppm 7.38-7.29 (m, 4H), 7.10 (d, J=5.9 Hz, 1H), 5.08 (s, 2H), 4.82 (s, 1H), 4.45 (q, J=6.2 Hz, 1H), 3.19 (q, J=6.4 Hz, 2H), 1.96-1.75 (m, 2H), 1.53-1.23 (m, 14H). OTOF/MS [C₂₀H₂₇F₃N₂O₅+H]+m/z 433.194 (experimental), 433.195 (calc'd).

TFA-Lys(H)-(OtBu)

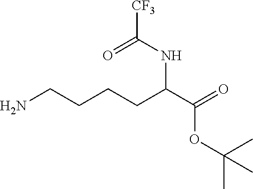 [SMILES: CC(C)(C)OC(=O)C(CCCCN)NC(=O)C(F)(F)F]

A solution of TFA-Lys-(Z)-(OtBu (22 g, 4.6/mmol) in ethanol (50 mL) was treated with ammonium formate (3.00 g, 46.7 mmol) and palladium on carbon (310 mg) under an atmosphere of N₂ gas for 16 h. The mixture was then filtered through a pad of celite and evaporated to dryness to yield a colourless oil that was used without further purification (1.66 g) OTOF/MS [C₁₂H₂₁F₃N₂O₃+H]⁺ m/z 299.158 (experimental), 299.158 (calc'd).

N-hydroxysuccinamydyl 4-(iodophenyl)butyrate

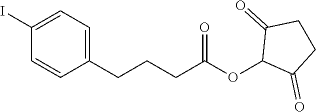 [SMILES: O=C(CCCc1ccc(I)cc1)OC1C(=O)CCC1=O]

A solution of 4(p-iodophenyl)butyric acid (2.09 g, 7.20 mmol), N-hydroxy succinimide (1.42 g, 12.3 mmol) and 1-ethyl-3-(3-dimethylaminopropyl)carbodiimide hydrochloride (1.78 g, 11.5 mmol) in acetonitrile (60 mL) was stirred at room temperature for 16 h. The solvent was then evaporated under reduced pressure to yield a colourless oil that solidified upon standing. The resulting solid was then dissolved in chloroform (50 mL), washed with saturated sodium bicarbonate solution (50 mL) followed by water (2×50 mL), then brine (50 mL). The organic fraction was then dried over Na₂SO₄, filtered, then evaporated to dryness to yield a colourless solid that was recrystallised from chloroform/pentane to yield a semi-crystalline colourless solid (2.13 g, 5.50 mmol, 76% yield). ¹H NMR (400 MHz; CDCl₃): δ_{H}/ppm 7.61 (d, J=8.1 Hz, 2H), 6.96 (d, J=8.1 Hz, 2H), 2.84 (s, 4H), 2.68 (t, J=7.6 Hz, 2H), 2.59 (t, J=7.3 Hz, 2H), 2.04 (quintet, J=7.5 Hz, 2H). OTOF/MS: [C₁₅H₁₅IO₄+H⁺]⁺ m/z 387.01 (experimental), 387.01 (calc'd).

TFA-Lys(IPB)-(OtBu)

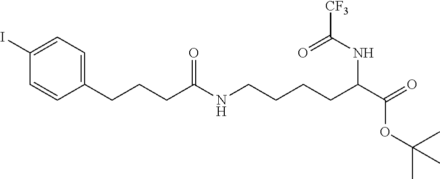 [SMILES: CC(C)(C)OC(=O)C(CCCCNC(=O)CCCc1ccc(I)cc1)NC(=O)C(F)(F)F]

A solution of TFA-Lys(H)-OtBu (1.60 g, 5.37 mmol if pure), N-hydroxysuccinamydyl 4-(iodophenyl)butyrate (2.46 g, 6.35 mmol) and DIPEA (0.9 mL) in dichloromethane was stirred at room temperature for 16 h. The reaction mixture was then washed with water (2×50 mL) followed by brine (2×50 mL) and the organic fraction collected, dried over Na₂SO₄, filtered and evaporated to dryness. The resulting residue was then purified by column chromatography (SiO₂, EtOAc in petroleum spirits, 0-50%) to yield a colourless residue (1.06 g, 1.85 mmol, 34% yield). ¹H NMR (400 MHz; CDCl₃): δ_{H}/ppm 7.59 (d, J=8.2 Hz, 2H), 7.13 (d, J=6.9 Hz, 1H), 6.92 (d, J=8.1 Hz, 2H), 5.49 (s, 1H), 4.43 (td, J=7.4, 4.8 Hz, 1H), 3.23 (7, J=6.3 Hz, 2H), 2.58 (t, J=7.5 Hz, 2H), 2.14 (t, J=7.5 Hz, 2H), 1.92 (dt, J=15.1, 7.4 Hz, 3H), 1.83-1.74 (m, 1H), 1.53 (dd, J=11.8, 4.9 Hz, 2H), 1.47 (s, 9H), 1.37-1.31 (m, 2H). OTOF/MS: [C₂₂H₃₀IN₂O₄+H⁺]⁺ m/z 571.128 (experimental), 571.128 (calc'd).

H-Lys(IPB)-(OtBu)

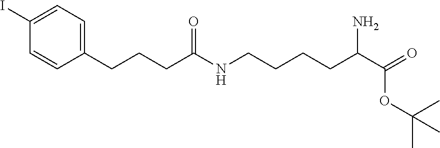 [SMILES: CC(C)(C)OC(=O)C(N)CCCCNC(=O)CCCc1ccc(I)cc1]

A mixture of TFA-Lys(IPB)-OtBu (393 mg, 0.69 mmol) and Na₂CO₃ (393 mg, 3.71 mmol) in H₂O/MeCN (50 mL) and stirred at room temperature for 16 h. The reaction mixture was then concentrated under reduced pressure and the mixture extracted with chloroform (2×50 mL). The combined organic fractions were then dried over Na₂SO₄, filtered and then concentrated to dryness to yield a colourless oil (280 mg, 0.59 mmol, 86% yield). ¹H NMR (400 MHz; CDCl₃): δ_{H}/ppm 7.54 (d, J=8.2 Hz, 2H), 6.88 (d, J=8.1 Hz, 2H), 5.83 (s, 1H), 3.25 (t, J=6.2 Hz, 1H), 3.18 (q, J=6.5 Hz, 2H), 2.54 (t, J=7.5 Hz, 2H), 2.09 (t, J=7.5 Hz, 2H), 1.88 (t, J=7.5 Hz, 2H), 1.80 (s, 2H), 1.49 (t, J=7.5 Hz, 2H), 1.42 (s, 9H), 1.36 (t, J=7.3 Hz, 2H). OTOF/MS: [C₂₀H₃₁IN₂O₃+H⁺]⁺ m/z 475.145 (experimental), 475.145 (calc'd).

(tBoc)₄₋₅sar(BBN)(ABG)—Method 1

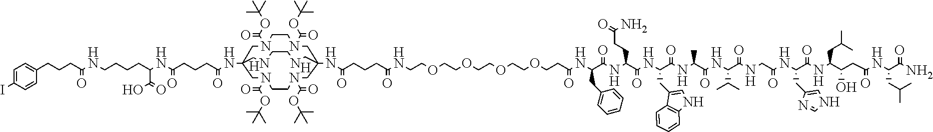

A mixture of (tBoc)₄/5sar(NHS)₂ (5 mg, 4.4 μmol), RM26 (6 mg, 4.4 μmol) and DIPEA (100 μg) in N-methylpyrrolidinone (1 mL) was heated to 70° C. with microwave radiation in a microwave vial for 15 min. To this was added H-Lys(IPB)-OH (9 mg, 22 μmol) and heated to 70° C. for 15 min with microwave irradiation. The addition of ether to the cooled mixture precipitated a colourless solid that was collected, dissolved in H₂O:MeCN (50:50, 5 mL) and purified by semi-preparative HPLC (20% B in A, 60 min). Fractions containing the desired product were collected and lyophilised to yield a colourless powder (1 mg, 0.04 μmol). OTOF/MS: [C₁₂₆H₁₉₈IN₂₅O₃₁+2H]²⁺ m/z 1343.695 (experimental), 1343.695 (calculated), [C₁₃₁H₂₀₆IN₂₅O₃₃+2H⁺]²⁺ m/z 1393.721 (experimental), 1393.721 (calculated). RP-HPLC Rt: 16.2 min.

sar(BBN)(ABG)

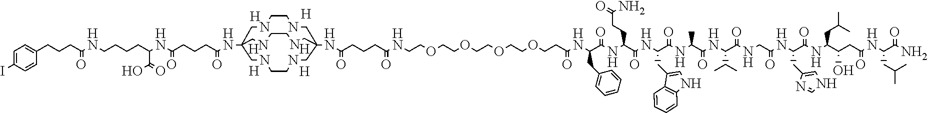

(tBoc)_{4/5}sar(ABG)(BBN) (1 mg, x mmol) was dissolved in TFA (1 mL) and incubated for 1 h at room temperature before volatiles were removed by evaporation under a stream of nitrogen gas. The resulting residue was then dissolved in H₂O/MeCN (50:50, 2 mL) and lyophilised to yield a colourless powder (0.25 mg, 0.11 μmol). OTOF/MS: [C₁₀₆H₁₆₆IN₂₅O₂₃+3H⁺]³⁺ m/z 762.726 (experimental), 762.730 (calc'd), [C₁₀₆H₁₆₆IN₂₅O₂₃+4H⁺]⁴⁺ m/z 572.296 (experimental), 572.299 (calc'd). RP-HPLC Rₜ: 12.3 min.

### Radiolabelling and TLC Analysis of Constructs

All constructs were incubated with ⁶⁴Cu at an excess of peptide in buffer (see Table 1 for reaction conditions) for 15-60 minutes. Samples of each solution were taken and mixed 1:1 with 50 mM EDTA. 5 μL of each solution was spotted on TLC paper (Agilent iTLC-SG Glass microfiber chromatography paper impregnated with silica gel) and run with 50:50 H₂O:ethanol. Plates were then imaged on an Eckert & Ziegler Mini-Scan and Flow-Count iTLC Reader. HPLC was also performed on samples for the first radiolabelling to validate the TLC results. All samples used for in vivo imaging were >95% labelling.

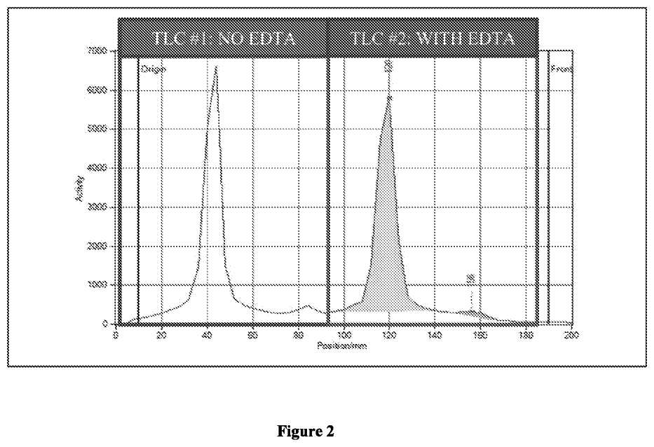

Control experiments were conducted to monitor the elution behaviour of free ⁶⁴Cu and ⁶⁴Cu bound to EDTA for quality control, as well as each sample also run with EDTA to check for radiopurity. A representative radioTLC image showing that all ⁶⁴Cu was bound to the dendrimers can be found in FIG. 2.

Peptides were radiolabelled in aqueous ethanol with a suitable buffer, as shown in Table 1. The use of a sodium phosphate (Na₂PO₄) buffer resulted in low radiochemical yield. Since ethanol can have a basic effect on aqueous/organic solvent mixtures, the use of ammonium acetate (NH₄OAc) with a lower pH was more effective.

TABLE 1
Summary of radiolabeling reactions
under different buffer conditions.

| | | % | % | |
|---|---|---|---|---|
| | Peptide | Labelled | Unbound | |
| Peptide | excess | peptide | Cu | Conditions |
| Sar(ABG)(BBN) | 400 | 10 | 90 | 50% EtOH in |
| | 400 | 3 | 97 | Na₂PO₄ buffer |
| | 400 | 12 | 88 | (0.1M, pH 6.2) |
| | 400 | 100 | 0 | 50% EtOH in |
| | 600 | 100 | 0 | NH₄OAc buffer |
| | | | | (0.1M, pH 5.5) |

### Cell Binding Studies

PC3 cells were seeded at a density of 5×104 cells per well in 24-well plates and incubated overnight with medium (RPMI 160 containing 10% fetal bovine serum and 1% streptomycin-penicillin). Approximately 200 kBq of radio-ligand was added to the medium, and the cells were incubated (in triplicate) for 15, 30, and 60 min at 25° C. At each time point, internalization was stopped by removing the medium and washing the cells twice with ice-cold PBS (pH 7.4, 0.5 mL). To remove the receptor-bound radioligand, an acid wash was carried out twice with ice-cold glycine buffer (0.1 M, pH 3.0, 1 mL) for 5 minutes. Cells were solubilized with NaOH (1 N, 2 mL) and the internalized fractions collected. The radioactivity of the supernatant, receptor-bound and internalized fractions were measured in a gamma-counter. Gamma counts were decay-corrected and converted to Becquerels, and the receptor-bound and internalized fractions represented as a percentage of applied activity per 105 cells.

### Animals

Healthy male Balb/c nude mice (˜18 g) from 8 weeks old were obtained from the ARC and used for this study. Mice were imported into the CAI animal holding facility and monitored for 1 week prior to the study in order to acclimatise to the environment prior to injection of cells. All animals were provided with free access to food and water before and during the imaging experiments which were approved by the University of Queensland Animal Ethics Committee (Approval #AIBN/CAI/105/19/ARC/NHMRC).

### Tumour Initiation and Growth

8-week-old male Balb/c nude mice were injected (27 G needle) subcutaneously with PC3 (2×106) cells in 50 μL of 50:50 matrigel and cells in phosphate buffered saline into the right flank of each mouse. There was no evidence of ulceration at the time of dosing; the animals were closely monitored and remained in good condition apart from the growth of tumours. The tumour growth was observed to be in line with expected timelines and good tumours were ultimately observed >80% of inoculated animals. Labelled peptides were injected via the tail vein (29 G needle; ˜2-3 MBq) and then mice were imaged using the Siemens Inveon PET-CT instrument at the various timepoints.

### Imaging Protocol

Mice were anaesthetised with isoflurane (IsoFlo, Abbott Laboratories) at a dose of 2% in a closed anaesthetic induction chamber. Mice were monitored using ocular and pedal reflexes to ensure deep anaesthesia. Once the mouse was deeply anesthetised, it was placed on an appropriate animal bed, where the anaesthetic air mixture (1%) was delivered to its nose and mouth through a nose cone. Physiological monitoring (respiratory using a sensor probe) was achieved throughout all experiments using an animal monitoring system (the BioVet™ system, m2m Imaging, Australia). Images were acquired using a Siemens Inveon PET-CT scanner following tail vein intravenous injection of the test articles.

The injection syringe was filled with the radioisotope solution (approximately 150 μL) and the activity in the syringe was measured using a dose calibrator (Capintec CRC-25) with a calibration factor of 35. The activity left in the syringe after the tail vein injection was measured using the same dose calibrator and the total volume injected in each mouse was calculated.

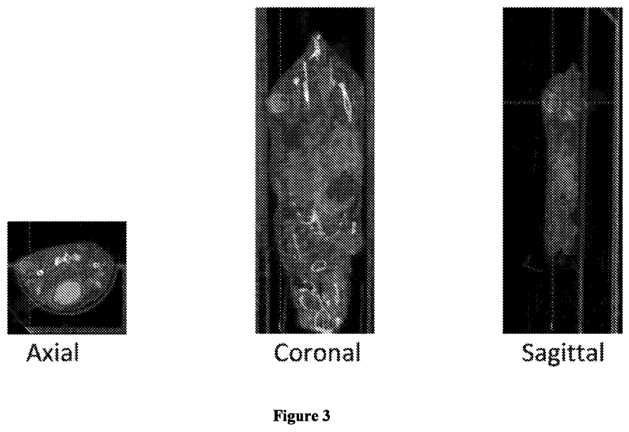

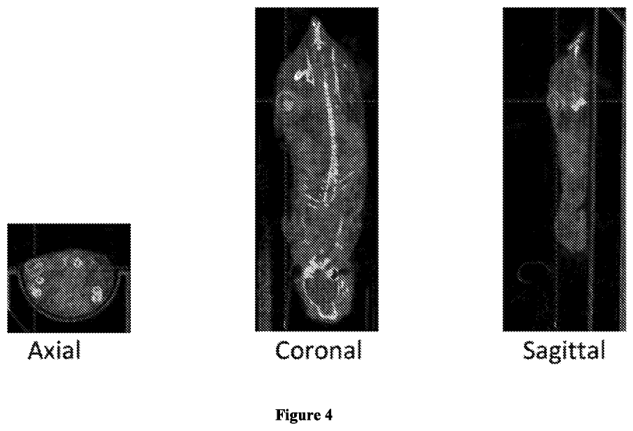

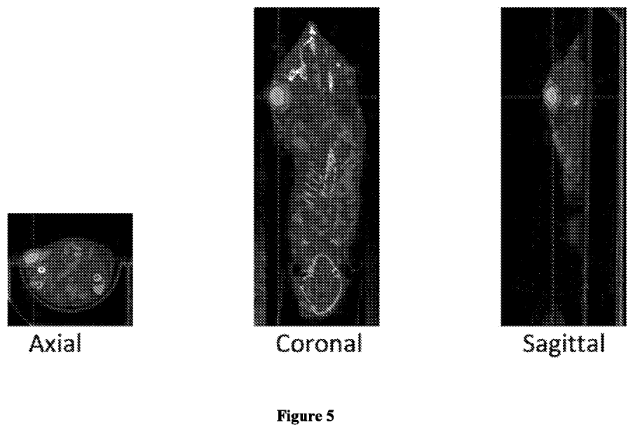

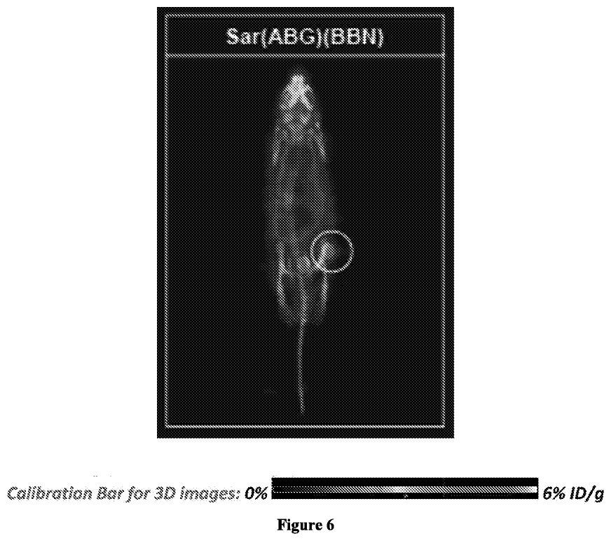

Calibration of the PET/CT scanner was performed with an in-house manufactured phantom containing a known activity of 68Ge solution as a radiation source. The mice were positioned on the scanner bed (n=4 per scan using a bed developed in-house) and micro-CT scans were acquired for anatomical co-registration. The CT images of the mice were acquired through an X-ray source with the voltage set to 80 kV and the current set to 500 μA. The scans were performed using 3600 rotation with 120 rotation steps with a low magnification and a binning factor of four. The exposure time was 230 ms with an effective pixel size of 106 μm. The total CT scanning process took approximately 15 minutes. The CT images were reconstructed using Feldkamp reconstruction software (Siemens). Following CT imaging, PET scans were acquired at, 1 hour, 4 hours and 24 hours after injection of the radiotracer (see FIGS. 3, 4 and 5 for images acquired after injection of Sar(ABG)(BBN)), using 30-90-minute static acquisitions. All images were static acquisitions wi The PET Images were reconstructed using an ordered-subset expectation maximization (OSEM2D) algorithm and analysed using the Inveon Research Workplace software (IRW 4.1) (Siemens) which allows fusion of CT and PET images and definition of regions of interest (ROIs). CT and PET datasets of each individual animal were aligned using IRW software (Siemens) to ensure good overlap of the organs of interest. Three dimensional ROIs were placed within the whole body, as well as all the organs of interest, such as heart, kidney, lungs, bladder, liver, spleen, pancreas and tumour, using morphologic CT information to delineate organs. Activity per voxel was converted to nCi/cm³ using a conversion factor obtained by scanning a cylindrical phantom filled with a known activity of ⁶⁴Cu to account for PET scanner efficiency. Activity concentrations were then expressed as percent of the decay-corrected injected activity per cm³ of tissue that can be approximate as percentage injected dose/g (% ID/g). Representative 3D images were also generated (see FIG. 6).

### Injected Dose (% ID/g) in Excised Organs

Organs were excised at 24 hours, with the percentage of injected dose of the administered compound determined by ex vivo gamma counting (Table 2). Values are averages across 4 mice.

The structures of the relevant compounds are as follows:

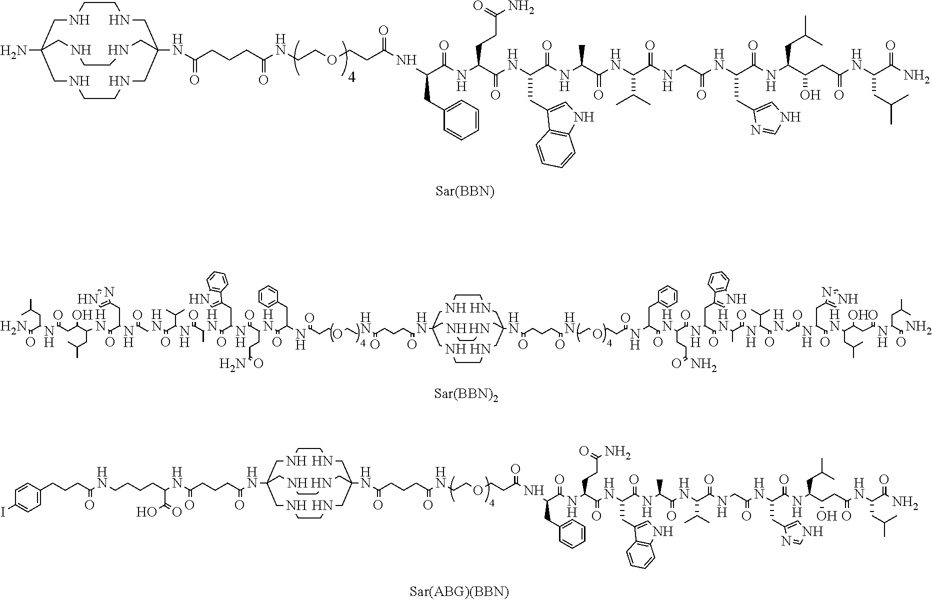

TABLE 2
Percentage of injected dose of Sar(BBN),
Sar(BBN)₂ and Sar(ABG)(BBN) in organs after
24 hours, as determined by ex vivo gamma counting.

| | Sar(BBN) | Sar(BBN)₂ | Sar(ABG)(BBN) |
|---|---|---|---|
| | (% ID/g) | (% ID/g) | (% ID/g) |
| Tumour | 4.09 ± 0.52 | 2.13 ± 0.86 | 22.30 ± 11.00 |
| Liver | 1.52 ± 0.18 | 16.80 ± 1.55 | 2.96 ± 0.30 |
| Spleen | 0.38 ± 0.06 | 1.47 ± 0.20 | 2.68 ± 0.33 |
| Kidneys | 3.55 ± 0.79 | 6.81 ± 0.67 | 4.29 ± 0.54 |
| Heart | 0.24 ± 0.02 | 0.21 ± 0.03 | 3.10 ± 0.53 |
| Lungs | 0.31 ± 0.08 | 0.36 ± 0.12 | 4.85 ± 0.93 |
| Blood | 0.13 ± 0.04 | 0.10 ± 0.05 | 7.22 ± 0.79 |
| Pancreas | 0.23 ± 0.02 | 0.84 ± 0.06 | 6.00 ± 1.17 |
| GI Tract | 0.30 ± 0.07 | 0.44 ± 0.04 | 1.93 ± 0.46 |
| Tail | 0.56 ± 0.87 | 0.28 ± 0.05 | 2.06 ± 0.30 |

### Post-Imaging Analysis of Tumour Uptake

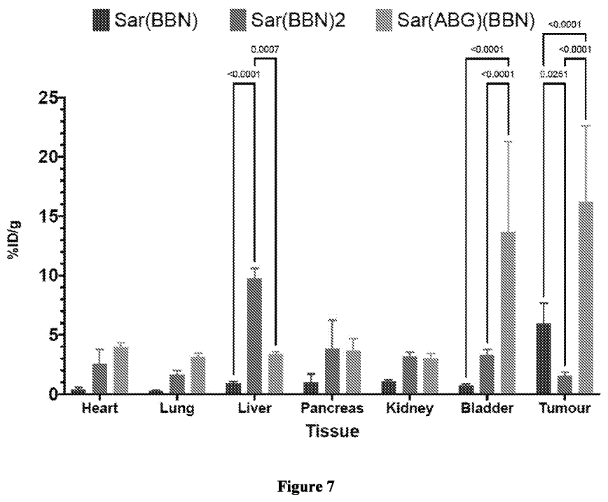

Regions of interest were drawn around the tumour margins (as delineated from the CT scan) and the concentration of peptide calculated for each mouse in the imaging study (based on % injected dose). Plots showing organ accumulation as measured by gamma counter in vivo and ex vivo after 24 hours were generated (see FIG. 7). Variability in quantitation between in vivo and ex vivo measurements arises due to region of interest (ROI) and background signal for the in vivo plots. Statistical significance was assigned as follows: ns P>0.05, *P≤0.05, **P≤0.01, ***P≤0.001, ****P≤0.0001 (see Table 3). In each case, Sar(ABG)(BBN) showed a statistically significant increase in tumour uptake, when compared to the other peptides without an albumin binding group on the compound.

TABLE 3
Statistical difference between accumulation of either Sar(BBN)
or Sar(BBN)₂ and Sar(ABG)(BBN) in tumours was determined.

| Comparison | Measurement | Summary | P-value |
|---|---|---|---|
| Sar(BBN) v Sar(ABG)(BBN) | in vivo | **** | <0.0001 |
| Sar(BBN)₂ v Sar(ABG)(BBN) | in vivo | **** | <0.0001 |
| Sar(BBN) v Sar(ABG)(BBN) | ex vivo | **** | <0.0001 |
| Sar(BBN)₂ v Sar(ABG)(BBN) | ex vivo | **** | <0.0001 |

What is claimed is:

1. A compound of Formula (I), a salt, complex, isomer, solvate or prodrug thereof: 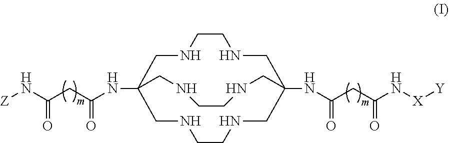
wherein:
each m is independently an integer from 1 to 10;
X is a linker having the formula 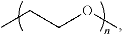
where n is an integer from 1 to 10;
Y is a group capable of binding to a biological receptor; and
Z is an albumin-binding group.

2. A compound of Formula (Ia), a salt, complex, isomer, solvate or prodrug thereof: 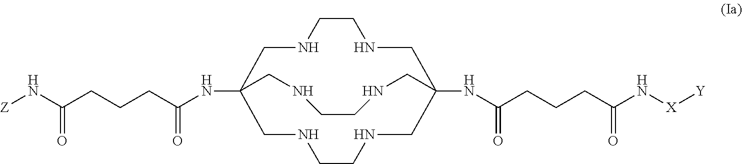
wherein:
X is a linker having the formula 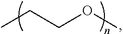
where n is an integer from 1 to 10;
Y is a group capable of binding to a biological receptor; and
Z is an albumin-binding group.

3. A compound according to claim 1, wherein X is a linker having the formula 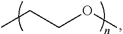
where n is an integer from 1 to 5.

4. A compound according to claim 1, wherein Z is an albumin-binding group of Formula (II): 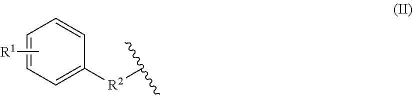
wherein:
R¹ is an optionally substituted alkyl group, optionally substituted alkoxy, halogen or CN group; and
R² is an optionally substituted C₂-C₁₆ alkylene group, wherein one or more alkylene units in the alkylene group may be replaced with a group selected from a urea, thiourea, amine, amide, carbonyl, heteroatom, polyethylene oxide or arylene group, wherein the arylene group may be optionally substituted.

5. A compound according to claim 1, wherein Z is an albumin-binding group of Formula (II′): 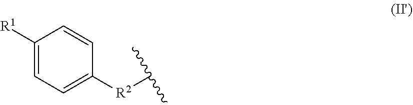
wherein R¹ and R² are as defined in claim 4.

6-7. (canceled)

8. A compound according to claim 1, wherein Z is an albumin binding group that is an optionally substituted C₂-C₂₀ alkyl group, wherein one or more alkylene units may be replaced with a group selected from a urea, thiourea, amine, amide, carbonyl, heteroatom or a polyethylene oxide group.

9. A compound according to claim 1, wherein Y is a protein, peptide, polypeptide, carbohydrate, oligonucleotide, liposome, oligosaccharide, antibody, steroid, nucleic acid, folic acid, vitamin B12, a fragment thereof or a derivative thereof.

10. (canceled)

11. A compound according to claim 1, wherein Y is selected from the group consisting of octreotate, octreotide, [Tyr³]-octreotate, bombesin, bombesin(7-14), peptides binding to prostate specific membrane antigen (PSMA), gastrin releasing peptide, penetratin, annexin V, TAT peptide, cyclic RGD, glucose, glucosamine, folic acid, neurotensin, neuropeptide Y, cholecystokinin (CCK) analogues, vasoactive intestinal peptide (VIP), substance P and alpha-melanocyte-stimulating hormone (MSH).

12. A compound according to claim 1, wherein the compound has one of the following structures:

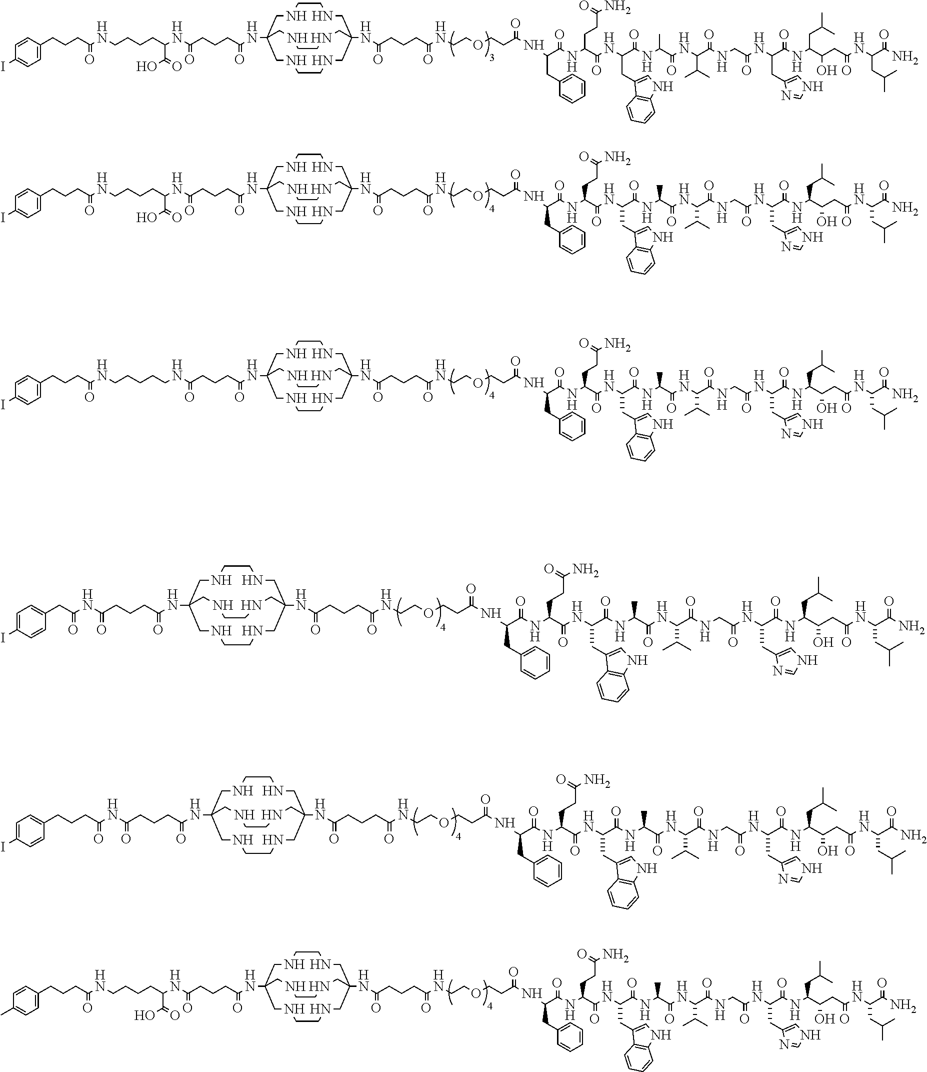

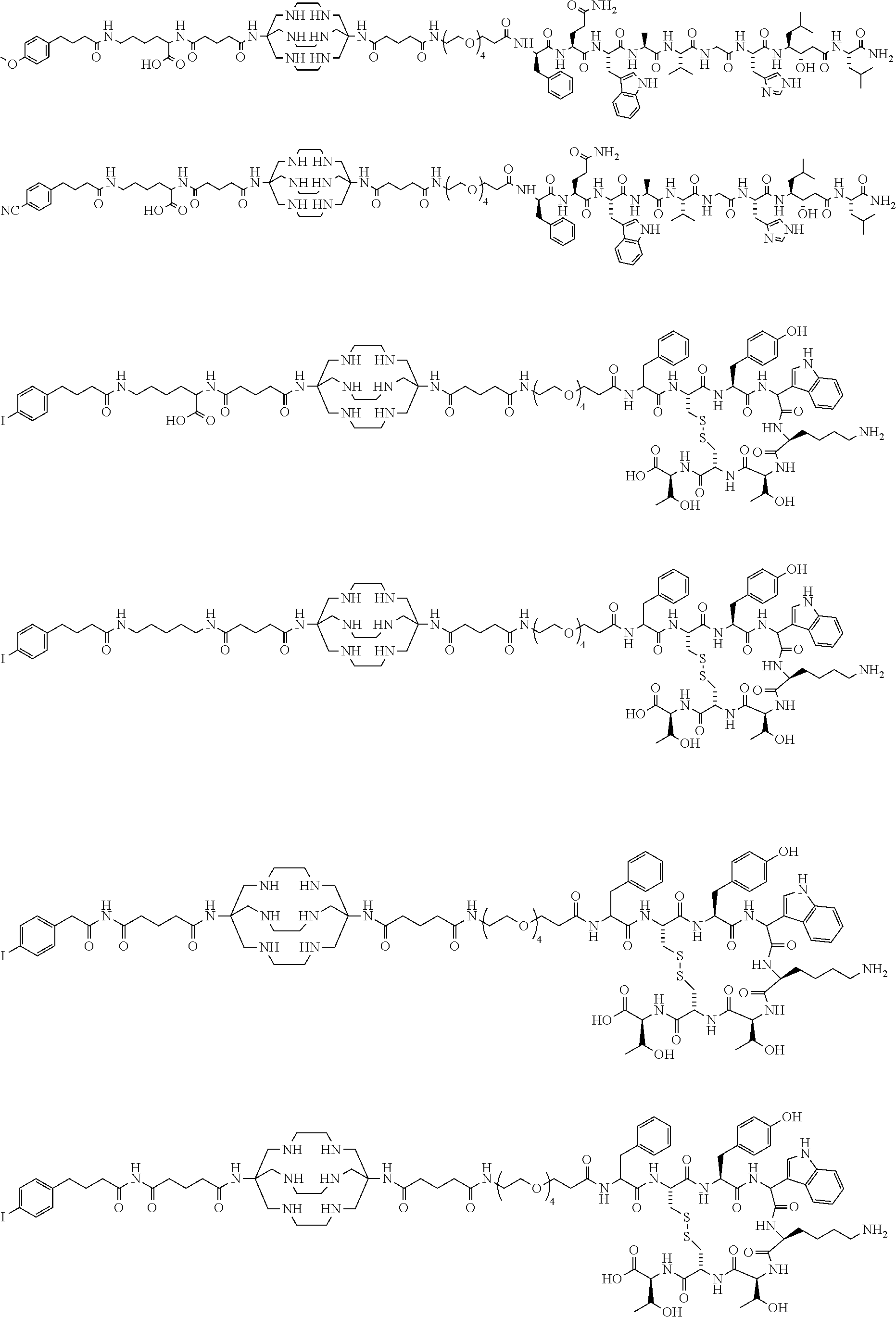

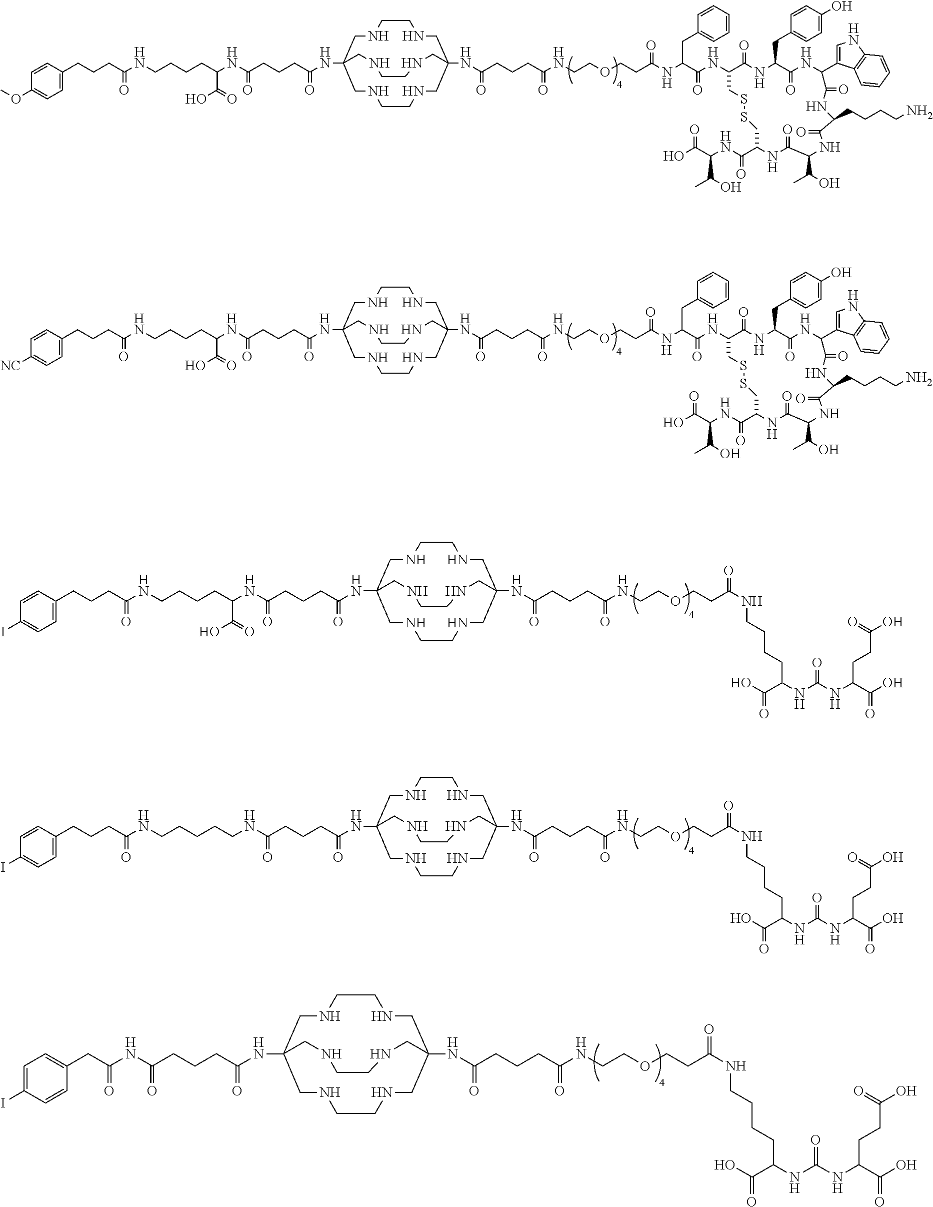

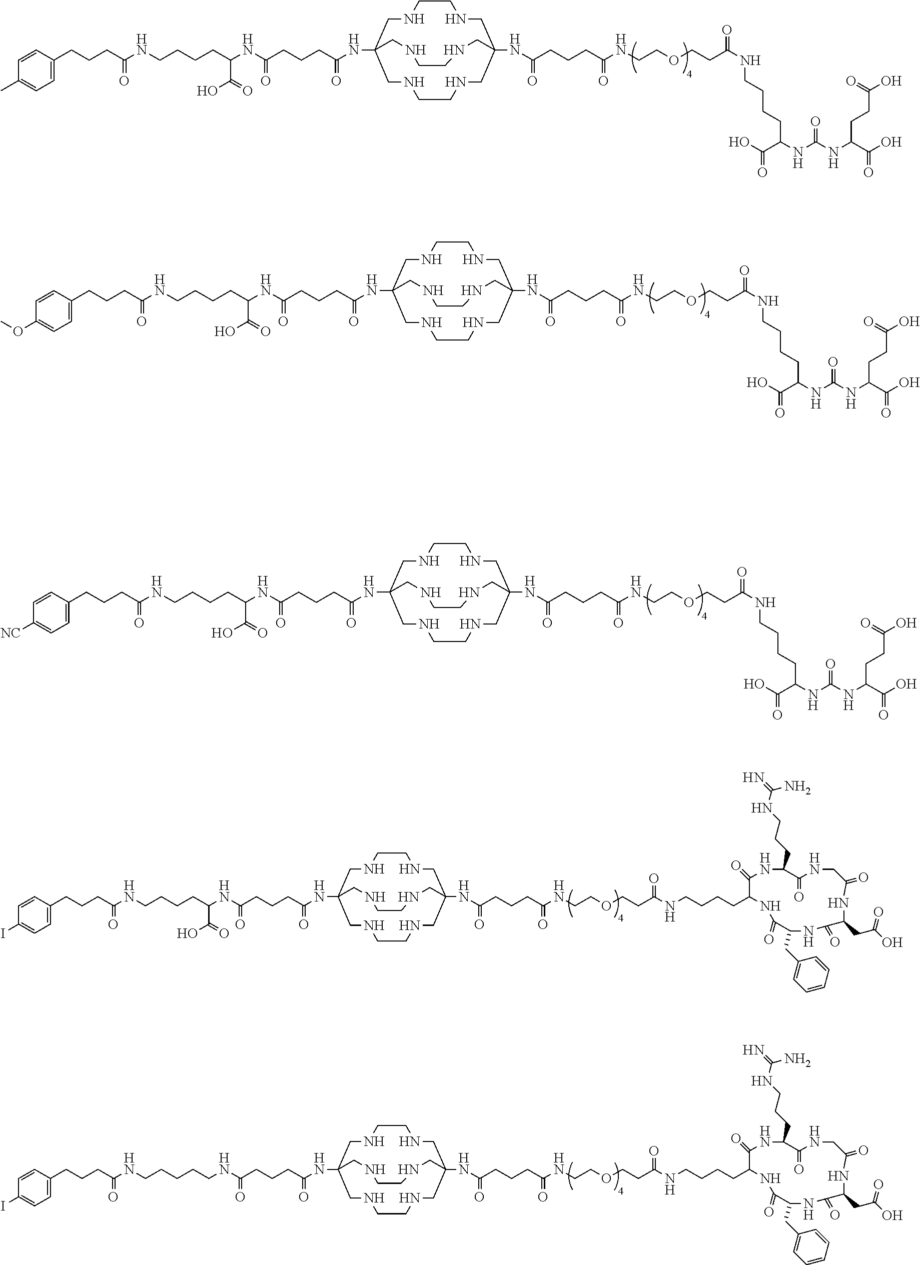

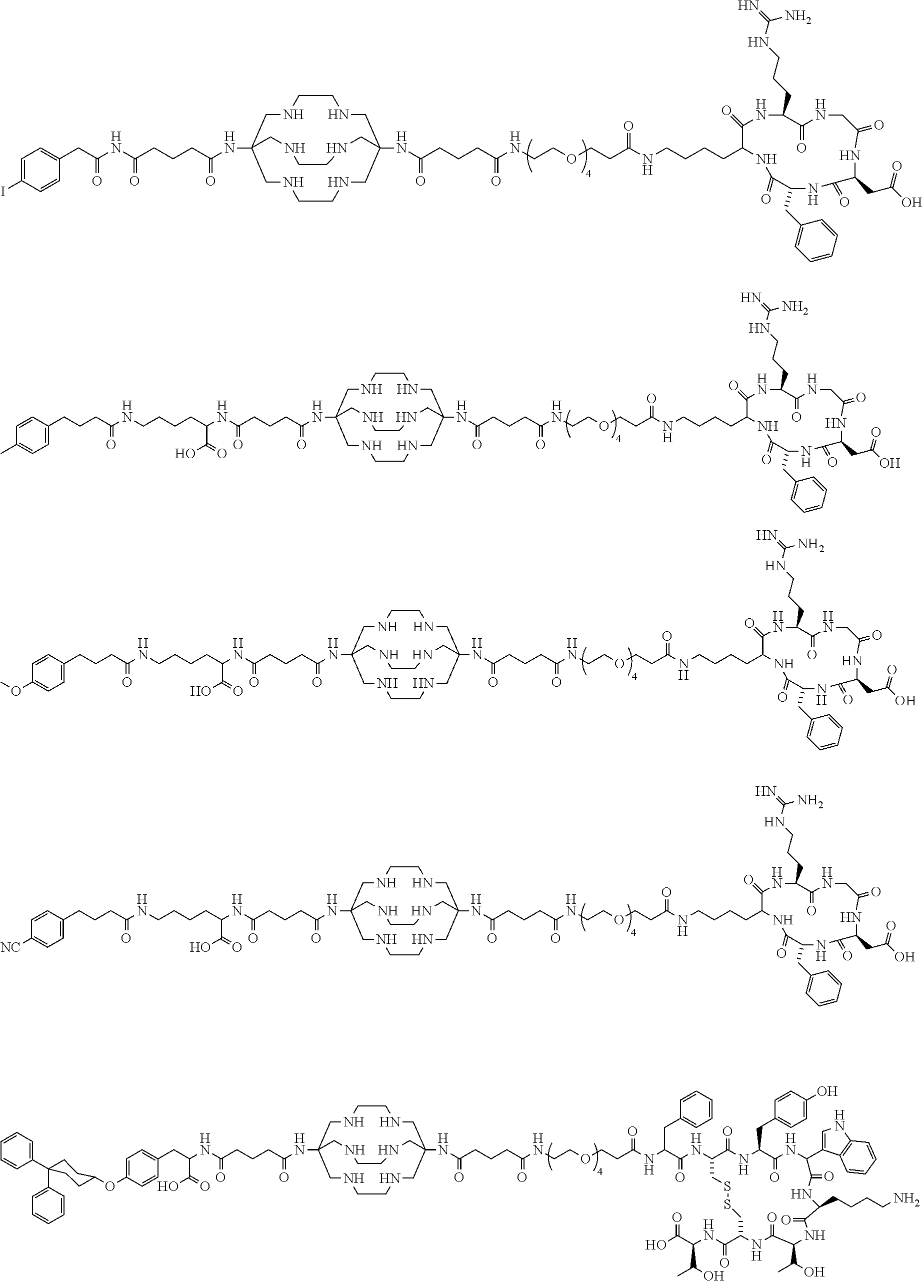

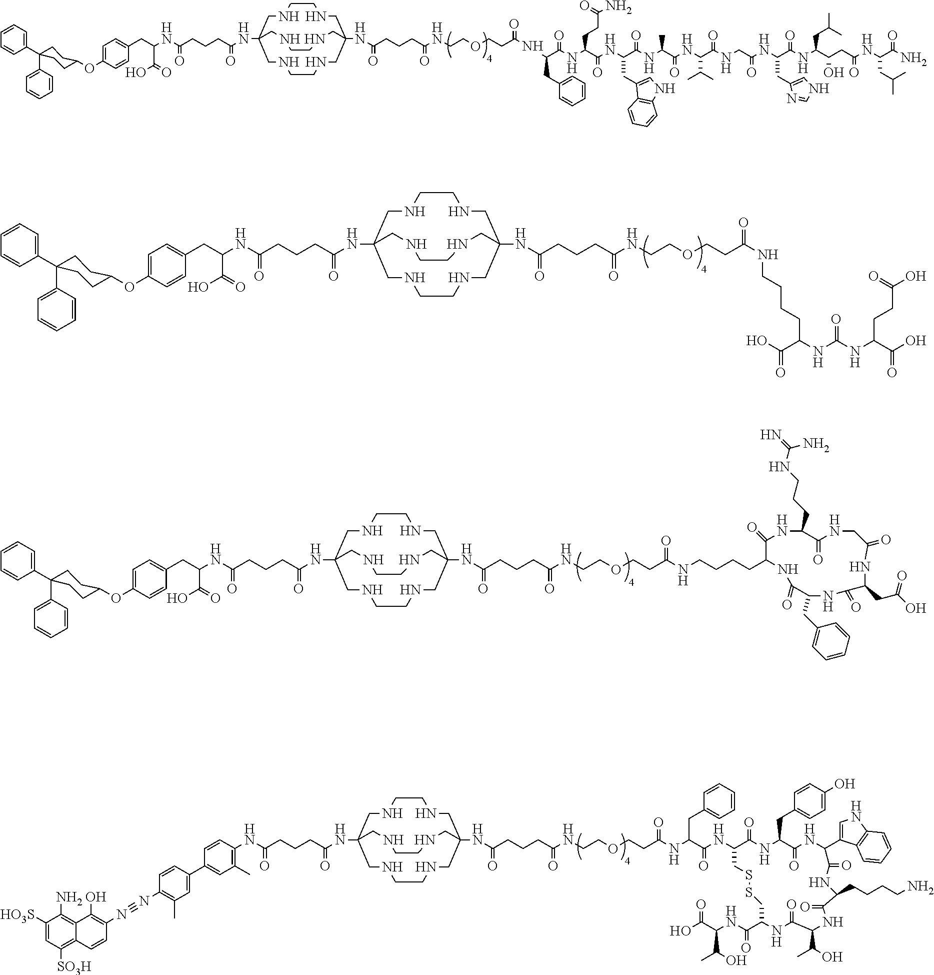

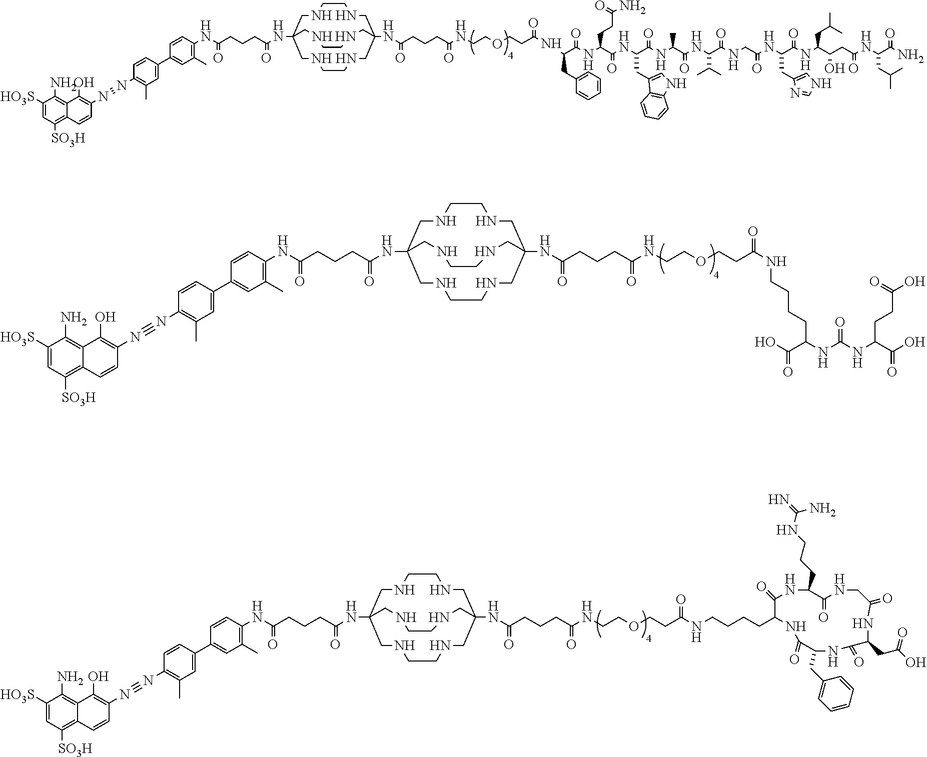

13. A compound according to claim 1, wherein the compound is coordinated with a metal ion.

14. A compound according to claim 13, wherein the metal ion is a radionuclide of a metal selected from the group consisting of Cu, Tc, Gd, Ga, In, Sc, Co, Re, Fe, Au, Ag, Rh, Pt, Bi, Cr, W, Ni, V, Ir, Zn, Cd, Mn, Ru, Pd, Hg, and Ti.

15. A compound according to claim 14, wherein the metal ion is a radionuclide of Cu.

16. A compound according to claim 15, wherein the metal ion is a radionuclide selected from the group consisting of ⁶⁰Cu, ⁶¹Cu, ⁶²Cu, ⁶⁴Cu and ⁶⁷Cu.

17. A composition comprising a compound of claim 1, and one or more pharmaceutically acceptable excipients.

18. A process for producing a compound of Formula (I) or a protected form thereof: 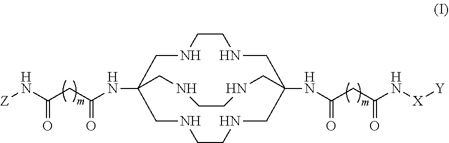
wherein m, X, Y and Z are as defined in claim 1, the process comprising the steps of:
i) coupling a compound of Formula (IV), or a salt, complex, isomer or solvate thereof, 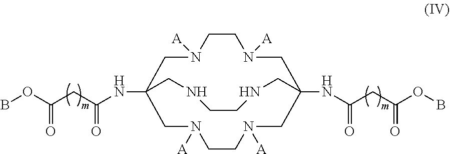
with a compound of Formula (V) or a salt thereof, 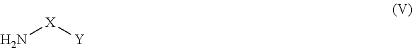
for a time and under conditions to give a compound of Formula (VI) or a salt thereof 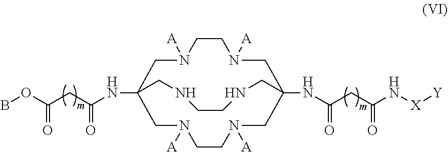
wherein A is a nitrogen-protecting group and B is an oxygen-protecting group;
ii) coupling a compound of Formula (VI) of step i) with a compound of Formula (VII) or a salt thereof:
H₂N—Z (VII)
for a time and under conditions to give a compound of Formula (VIII) or a salt thereof

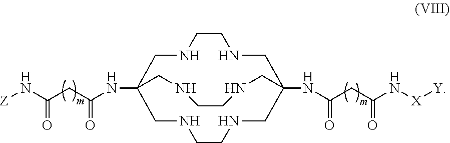

19. A process according to claim 18, wherein the compound of Formula (I) has the structure of Formula (Ia):

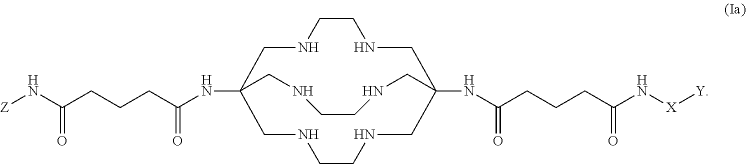

20. (canceled)

21. A compound of Formula (I), a salt, complex, isomer, solvate or prodrug thereof produced according to a process as defined in claim 18: 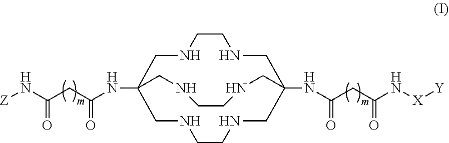
wherein:
each m is independently an integer from 1 to 10;
X is a linker having the formula 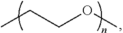
where n is an integer from 1 to 10;
Y is a group capable of binding to a biological receptor; and
Z is an albumin-binding group.

22. A method for treating a cancer in a subject, the method comprising administering to a subject in need thereof a compound as defined in claim 14.

23. A method according to claim 22, wherein the cancer includes a malignant, cancerous or pre-cancerous cell growth, or a leukemia, solid tumour or carcinoma, melanoma, or colon, lung, ovarian, skin, breast, pancreas, pharynx, brain, prostate, CNS, or renal cancer.

24. A method of radioimaging a subject, the method comprising administering to a subject in need thereof a compound as defined in claim 14.

25-27. (canceled)
